(19) United States
(12) Patent Application Publication
(10) Pub. No.: US 2023/0256025 A1
(43) Pub. Date: Aug. 17, 2023

(54) TREATMENT OF INTERVERTEBRAL DISC DEGENERATION AND DISCOGENIC BACK PAIN

(71) Applicant: KOLON TISSUEGENE, INC., Rockville, MD (US)

(72) Inventors: Moon Jong NOH, Rockville, MD (US); Huan T. Tran, Rockville, MD (US)

(73) Assignee: KOLON TISSUEGENE, INC., Rockville, MD (US)

(21) Appl. No.: 18/134,383

(22) Filed: Apr. 13, 2023

Related U.S. Application Data
(63) Continuation-in-part of application No. 17/092,779, filed on Nov. 9, 2020.
Application No. 17/092,779 is a continuation of application No. PCT/US20/25705, filed on Mar. 30, 2020.
(60) Provisional application No. 62/826,676, filed on Mar. 29, 2019.

(51) Int. Cl. A61K 35/35; A61K 35/22; A61K 35/36; A61P 29/00; C07K 14/495

(52) U.S. Cl. CPC A61K 35/35; A61K 35/22; A61K 35/36; A61P 29/00; C07K 14/495

(57) ABSTRACT

A method for preventing and treating back pain including discogenic back pain, which includes administering a mixed cell composition containing mammalian connective tissue cells and mammalian cells expressing TGF-β1 into the intervertebral disc defect site.

### CROSS REFERENCE TO RELATED APPLICATIONS

This application is a continuation-in-part of U.S. patent application Ser. No. 17/092,779 filed Nov. 9, 2020, which is a 371 National Stage Application of PCT/US2020/025705 filed Mar. 30, 2020, which claims priority from U.S. Provisional Application No. 62/826,676 filed Mar. 29, 2019.

### BACKGROUND

### Field

The present invention relates to prevention or retardation of intervertebral disc degeneration. The present application also relates to treating degenerating disc by preventing or retarding intervertebral disc degeneration. The present invention also relates to methods of using chondrocytes for introduction into injured intervertebral disc region and preventing or retarding degeneration of the intervertebral disc. The present invention also relates to a method of introducing at least one gene encoding a member of the transforming growth factor β superfamily into at least one mammalian cell for use in preventing or retarding degeneration of intervertebral disc in the mammalian host. The present invention also relates to a method of using a mixture of chondrocytes and mammalian cells expressing a gene encoding a member of the transforming growth factor β superfamily into injured intervertebral disc region and preventing or retarding degeneration of the intervertebral disc.

The present application also relates to prevention and treatment of back pain, such as low back pain associated with degeneration of intervertebral discs. The present application also relates to methods of using chondrocytes for introduction into injured intervertebral disc region and preventing or treating low back pain. The present invention also relates to a method of introducing at least one gene encoding a member of the transforming growth factor β superfamily into at least one mammalian cell for use in preventing or treating low back pain. The present invention also relates to a method of using a mixture of chondrocytes and mammalian cells expressing a gene encoding a member of the transforming growth factor β superfamily into intervertebral disc region and preventing or treating the low back pain, such as pain associated with degeneration of the intervertebral disc.

### SUMMARY

In one aspect, the present invention is directed to a method for preventing or retarding degeneration of intervertebral disc at an intervertebral disc defect site, which includes injecting a mammalian connective tissue cell into the intervertebral disc defect site. In another aspect, the present invention is directed to a method for preventing or treating chronic low back pain. In some aspects, the chronic low back pain is discogenic low back pain. In some aspects, the discogenic low back pain is associated with or caused by degeneration of an intervertebral disc. The process preferably does not use a scaffolding or any supporting structure for the cells. That is, according to aspects of the present invention, the mammalian connective tissue cells are free of a scaffold or a three-dimensional support material that is pre-formed and shaped to accommodate the intended application, and the mammalian connective tissue cells do not contain deposits of cells formed by, for example, bioprinting. Preferably, non-transfected chondrocyte or fibroblast is used, and the subject is preferably a human being. If a chondrocyte is being used, the chondrocyte is preferably a non-disc chondrocyte or juvenile chondrocyte, meaning that the cells are isolated from a child who is less than two years old. In other aspects, the chondrocyte may be primed chondrocytes. In particular, the connective tissue cell may be allogeneic relative to the mammalian subject sought to be treated.

Transfected or transduced mammalian cells as discussed above may include epithelial cells, preferably human epithelial cells, or human embryonic kidney 293 cells, also referred to as HEK-293 or 293 cells.

In one aspect, the present invention relates to methods of using allogeneic juvenile chondrocytes or allogeneic non-disc chondrocytes for introduction into injured intervertebral disc region and preventing or retarding degeneration of the intervertebral disc.

In one aspect, the present invention relates to methods of using allogeneic juvenile chondrocytes or allogeneic non-disc chondrocytes for introduction into injured intervertebral disc region for preventing or treating the pain associated with degeneration of the intervertebral disc.

In one aspect, the present invention is used to prevent or retard further degeneration of an area in the intervertebral disc that has been injured, torn or herniated.

In one aspect, the present invention is used to prevent or treat the pain caused by degeneration of an area in the intervertebral disc that has been injured, torn or herniated.

In another aspect, the invention is directed to a method for preventing or retarding the degeneration of intervertebral disc at an intervertebral disc defect site of a mammal, which method includes a) inserting a gene or a nucleic acid sequence encoding a protein having intervertebral disc regenerating function into a mammalian cell, and b) injecting the mammalian cell into the intervertebral disc defect site. The process preferably does not use a scaffolding or any supporting structure for the cells. In this method, the protein may belong to TGF-β superfamily, such as TGF-β, and preferably TGF-β1.

It is to be understood that whenever a reference is made to a gene encoding a protein having intervertebral disc regenerating function being transfected, transduced or inserted into a cell, another nucleic acid encoding said protein may be substituted for said gene. Such nucleic acid may be, for example, a coding region of a gene, an intron sequence, a recombinant sequence or a synthetic sequence.

In another aspect, the invention is directed to a method for preventing or treating the pain at an intervertebral disc defect site of a mammal, which method includes a) inserting a gene encoding a protein having intervertebral disc regenerating function into a mammalian cell, and b) transplanting the mammalian cell into the intervertebral disc defect site. The process preferably does not use a scaffolding or any supporting structure for the cells. The mammalian cells are free of a scaffold or a three-dimensional support material that is pre-formed and shaped to accommodate the intended application, and the mammalian cells do not contain deposits of cells formed by, for example, bioprinting. In this method, the protein may belong to TGF-β superfamily, such as TGF-β, and preferably TGF-β1.

Transfected mammalian cells as discussed above may include epithelial cells, preferably human epithelial cells, or human embryonic kidney 293 cells, also referred to as HEK-293 or 293 cells. In some aspects, the mammalian cell is a GP2-293 packaging cells, also referred to as a GP2-293 cells.

It is to be understood that when cells transfected with a gene or a nucleic acid sequence encoding a protein having intervertebral disc regenerating function are discussed herein, cells that are transduced with said gene or a nucleic acid sequence, or cells that have said gene or nucleic acid sequence inserted into their genome by other means or that express said exogenous gene or nucleic acid sequence may be substituted for said transfected cells.

In yet another aspect, the invention is directed to method for preventing or retarding degeneration of intervertebral disc at an intervertebral disc defect site of a mammal, which includes a) inserting a gene encoding a protein having intervertebral disc regenerating function into a first mammalian cell to give transfected mammalian cell, and b) transplanting a mixture of the transfected mammalian cell of a) and unmodified second mammalian cell that is a connective tissue cell into the intervertebral disc defect site. The process preferably does not use a scaffolding or any supporting structure for the cells. The first and the second mammalian cells are free of a scaffold or a three-dimensional support material that is pre-formed and shaped to accommodate the intended application, and the mammalian cells do not contain deposits of cells formed by, for example, bioprinting. In this method, the protein may belong to TGF-β superfamily, such as TGF-β, and preferably TGF-β1. In this method, the first mammalian cells may be epithelial cells, preferably human epithelial cells, or human embryonic kidney 293 cells, also referred to as HEK-293 or 293 cells. In some aspects, the mammalian cells are GP2-293 packaging cells, also referred to as GP2-293 cells.

In yet another aspect, the invention is directed to a method for preventing or treating pain at an intervertebral disc defect site of a mammal, which includes a) inserting a gene encoding a protein having an intervertebral disc regenerating function into a first mammalian cell to obtain a transfected mammalian cell, and b) transplanting a mixture of the mammalian cell of a) and unmodified second mammalian cell that is a connective tissue cell into the intervertebral disc defect site. The process preferably does not use a scaffolding or any supporting structure for the cells. The first and the second mammalian cells are free of a scaffold or a three-dimensional support material that is pre-formed and shaped to accommodate the intended application, and the mammalian cells do not contain deposits of cells formed by, for example, bioprinting. In this method, the protein may belong to TGF-β superfamily, such as TGF-β, and preferably TGF-β1.

The first transfected mammalian cells as discussed above may include epithelial cells, preferably human epithelial cells, or human embryonic kidney 293 cells, also referred to as HEK-293 or 293 cells. In some aspects, the mammalian cells are GP2-293 packaging cells. The GP2-293 packaging cells are derived from HEK-293 cells which were modified to stably express retroviral gag and pol proteins. The GP2-293 packaging cells may be irradiated. The GP2-293 packaging cells are transduced to express a gene encoding a protein belonging to the TGF-β superfamily, such as TGF-β, and preferably TGF-β1.

In some aspects, the gene or a nucleic acid sequence encoding a protein belonging to the TGF-β superfamily may encode a mammalian protein, more specifically a human protein. In some aspects, the protein may be a recombinant protein.

The second mammalian cells may be connective tissue cells such as chondrocytes or fibroblasts. In the case of chondrocytes, the chondrocytes may be non-disc chondrocytes or juvenile chondrocytes. In particular, the chondrocytes for the second mammalian connective tissue cells may be primed chondrocytes. In another aspect, either or both of the first or second connective tissue cells may be allogeneic relative to the mammalian subject or to each other.

### BRIEF DESCRIPTION OF THE DRAWINGS

FIGS. 1A-1F show a slowing, retardation or prevention of degeneration of injured disc. (A) shows magnetic resonance imaging (MRI) radiograph of rabbit spine pre-surgery; (B) shows MRI radiograph of a rabbit spine four (4) weeks after surgery in which (i) the disc at L1/2 was injured and TGF-β1-producing 293 cells were injected, (ii) no puncture and no treatment is seen at spine locus L2/3, and (iii) disc at L3/4 was injured and mixture of TGF-β1-producing 293 cells and untransduced human chondrocytes in 1:3 ratio were injected; arrows point to L1/2 and L3/4 disc region. (C) shows MRI radiograph of a rabbit spine eight (8) weeks after surgery in which (i) the disc at L1/2 was injured and TGF-β1-producing 293 cells were injected, (ii) no puncture and no treatment control at spine locus L2/3, and (iii) disc at L3/4 was injured and mixture of TGF-β1-producing 293 cells and untransduced human chondrocytes in 1:3 ratio were injected; arrows point to L1/2 and L3/4 disc region. (D) shows X-ray radiograph of the rabbit described in (A) above, which is used to obtain a disc height index of the intervertebral disc to measure its morphology, its level of degeneration or regeneration. (E) shows X-ray radiograph of the rabbit described in (B) above, which is used to obtain a disc height index of the intervertebral disc. (F) shows X-ray radiograph of the rabbit described in (C) above, which is used to obtain a disc height index of the intervertebral disc. Mixed cell treatment in particular, has an intervertebral anti-degenerating effect.

FIGS. 2A-2F show a slowing, retardation or prevention of degeneration of injured disc. (A) shows MRI radiograph of rabbit spine pre-surgery; (B) shows MRI radiograph of a rabbit spine four (4) weeks after surgery in which (i) the disc at L1/2 was injured and TGF-β1-producing 293 cells were injected, (ii) no puncture and no treatment control at spine locus L2/3, and (iii) disc at L3/4 was injured and mixture of TGF-β1-producing 293 cells and untransduced human chondrocytes in 1:3 ratio were injected; arrows point to L1/2 and L3/4 disc region. (C) shows MRI radiograph of a rabbit spine eight (8) weeks after surgery in which (i) the disc at L1/2 was injured and TGF-β1-producing 293 cells were injected, (ii) no puncture and no treatment is seen at spine locus L2/3, and (iii) disc at L3/4 was injured and mixture of TGF-β1-producing 293 cells and untransduced human chondrocytes in 1:3 ratio were injected; arrows point to L1/2 and L3/4 disc region. (D) shows X-ray radiograph of the rabbit described in (A) above, which is used to obtain a disc height index of the intervertebral disc to measure its morphology, its level of degeneration or regeneration. (E) shows X-ray radiograph of the rabbit described in (B) above, which is used to obtain a disc height index of the intervertebral disc. (F) shows X-ray radiograph of the rabbit described in (C) above, which is used to obtain a disc height index of the intervertebral disc. Mixed cell treatment in particular, has an intervertebral anti-degenerating effect.

FIGS. 3A-3D show a slowing, retardation or prevention of degeneration of injured disc. (A) shows MRI radiograph of rabbit spine pre-surgery; (B) shows MRI radiograph of a rabbit spine four (4) weeks after surgery in which (i) the disc at L1/2 was injured and TGF-β1-producing 293 cells were injected, (ii) no puncture and no treatment control at spine locus L2/3, and (iii) disc at L3/4 was injured and mixture of TGF-β1-producing 293 cells and untransduced human chondrocytes in 1:3 ratio were injected; arrows point to L1/2 and L3/4 disc region. (C) shows X-ray radiograph of the rabbit described in (A) above, which is used to obtain a disc height index of the intervertebral disc to measure its morphology, its level of degeneration or regeneration. (D) shows X-ray radiograph of the rabbit described in (B) above, which is used to obtain a disc height index of the intervertebral disc. Mixed cell treatment in particular, has an intervertebral anti-degenerating effect.

FIGS. 4A-4D show a slowing, retardation or prevention of degeneration of injured disc. (A) shows MRI radiograph of rabbit spine pre-surgery; (B) shows MRI radiograph of a rabbit spine four (4) weeks after surgery in which (i) the disc at L1/2 was injured and mixture of TGF-β1-producing 293 cells and untransduced human chondrocytes in 1:3 ratio were injected, (ii) no puncture and no treatment control at spine locus L2/3, and (iii) disc at L3/4 was injured and TGF-β1-producing 293 cells were injected; arrows point to L1/2 and L3/4 disc regions. (C) shows X-ray radiograph of the rabbit described in (A) above, which is used to obtain a disc height index of the intervertebral disc to measure its morphology, its level of degeneration or regeneration. (D) shows X-ray radiograph of the rabbit described in (B) above, which is used to obtain a disc height index of the intervertebral disc. TGF-β1-producing 293 cells treatment in particular, has an intervertebral anti-degenerating effect.

FIGS. 5A-5D show a slowing, retardation or prevention of degeneration of injured disc. (A) shows MRI radiograph of rabbit spine pre-surgery; (B) shows MRI radiograph of a rabbit spine four (4) weeks after surgery in which (i) the disc at L1/2 was injured and mixture of TGF-β1-producing 293 cells and untransduced human chondrocytes in 1:3 ratio were injected, (ii) no puncture and no treatment control at spine locus L2/3, and (iii) disc at L3/4 was injured and TGF-β1-producing 293 cells were injected; arrows point to L1/2 and L3/4 disc regions. (C) shows X-ray radiograph of the rabbit described in (A) above, which is used to obtain a disc height index of the intervertebral disc to measure its morphology, its level of degeneration or regeneration. (D) shows X-ray radiograph of the rabbit described in (B) above, which is used to obtain a disc height index of the intervertebral disc. TGF-β1-producing 293 cells treatment and mixed cell treatments in particular, have an intervertebral anti-degenerating effect.

FIGS. 6A-6D show a slowing, retardation or prevention of degeneration of injured disc. (A) shows MRI radiograph of rabbit spine pre-surgery; (B) shows MRI radiograph of a rabbit spine four (4) weeks after surgery in which (i) the disc at L1/2 was injured and cell culture media DMEM was injected, (ii) no puncture and no treatment control at spine locus L2/3, and (iii) disc at L3/4 was injured and untransduced chondrocytes were injected; arrows point to L1/2 and L3/4 disc regions. (C) shows X-ray radiograph of the rabbit described in (A) above, which is used to obtain a disc height index of the intervertebral disc to measure its morphology, its level of degeneration or regeneration. (D) shows X-ray radiograph of the rabbit described in (B) above, which is used to obtain a disc height index of the intervertebral disc. Untransduced chondrocytes treatment has an intervertebral anti-degenerating effect.

FIGS. 7A-7F show a slowing, retardation or prevention of degeneration of injured disc. (A) shows MRI radiograph of rabbit spine pre-surgery; (B) shows MRI radiograph of a rabbit spine four (4) weeks after surgery in which (i) the disc at L1/2 was injured and cell culture media DMEM was injected, (ii) no puncture and no treatment control at spine locus L2/3, and (iii) disc at L3/4 was injured and untransduced chondrocytes were injected; arrows point to L1/2 and L3/4 disc regions. (C) shows MRI radiograph of a rabbit spine eight (8) weeks after surgery in which (i) the disc at L1/2 was injured and cell culture media DMEM was injected, (ii) no puncture and no treatment control at spine locus L2/3, and (iii) disc at L3/4 was injured and untransduced chondrocytes were injected; arrows point to L1/2 and L3/4 disc regions. (D) shows X-ray radiograph of the rabbit described in (A) above, which is used to obtain a disc height index of the intervertebral disc to measure its morphology, its level of degeneration or regeneration. (E) shows X-ray radiograph of the rabbit described in (B) above, which is used to obtain a disc height index of the intervertebral disc. (F) shows X-ray radiograph of the rabbit described in (C) above, which is used to obtain a disc height index of the intervertebral disc. Untransduced chondrocytes treatment has an intervertebral anti-degenerating effect.

FIGS. 8A-8F show a slowing, retardation or prevention of degeneration of injured disc. (A) shows MRI radiograph of rabbit spine pre-surgery; (B) shows MRI radiograph of a rabbit spine four (4) weeks after surgery in which (i) the disc at T12/L1 was injured by needle puncture and no injection, (ii) no puncture and no treatment control at spine locus L1/2, and (iii) disc at L2/3 was injured and untransduced chondrocytes were injected; arrows point to T12/L1 and L2/3 disc regions. (C) shows MRI radiograph of a rabbit spine eight (8) weeks after surgery in which (i) the disc at T12/L1 was injured by needle puncture and no injection, (ii) no puncture and no treatment control at spine locus L1/2, and (iii) disc at L2/3 was injured and untransduced chondrocytes were injected; arrows point to T12/L1 and L2/3 disc regions. (D) shows X-ray radiograph of the rabbit described in (A) above, which is used to obtain a disc height index of the intervertebral disc to measure its morphology, its level of degeneration or regeneration. (E) shows X-ray radiograph of the rabbit described in (B) above, which is used to obtain a disc height index of the intervertebral disc. (F) shows X-ray radiograph of the rabbit described in (C) above, which is used to obtain a disc height index of the intervertebral disc. Untransduced chondrocytes treatment has an intervertebral anti-degenerating effect.

FIGS. 9A-9D show a slowing, retardation or prevention of degeneration of injured disc. (A) shows MRI radiograph of rabbit spine pre-surgery; (B) shows MRI radiograph of a rabbit spine eight (8) weeks after surgery in which (i) the disc at L2/3 was injured and cell culture media DMEM was injected, (ii) no puncture and no treatment control at spine locus L3/4, and (iii) disc at L4/5 was injured and primed chondrocytes were injected; arrows point to L2/3 and L4/5 disc regions. (C) shows X-ray radiograph of the rabbit described in (A) above, which is used to obtain a disc height index of the intervertebral disc to measure its morphology, its level of degeneration or regeneration. (D) shows X-ray radiograph of the rabbit described in (B) above, which is used to obtain a disc height index of the intervertebral disc. Primed chondrocyte treatment has an intervertebral anti-degenerating effect.

FIG. 10 shows experimental design of the study described in Example VI. Time schedules for the dynamic weight bearing tests (DWB) and withdrawal tests in hindlimbs (vFF) are illustrated. Intervertebral disc degenerative (IVDD) animal model was induced using monosodium iodoacetate (MIA). Application of the mixed cells composition (n=7) or vehicle (n=8) was performed 14 days after generating MIA-induced IVDD or LBP animal model. Animals that underwent sham surgery were not injected (n=10). In this and following figures, treatment with MIA and the mixed cell composition is designated as MIA-drug; treatment with MIA and vehicle is designated as MIA-veh; sham surgery and no subsequent treatment is designated as SHAM. The specific mixed cell compositions used in the examples are sometimes referred to in figures as TG-C drug.

FIG. 11 shows the results of the weight bearing test described in Example VI. The HIND-FORE weight bearing difference in the MIA-induced LBP group was reduced from 11-12% to 2-3% due to the development of LBP. However, application of the mixed cell composition (n=7) significantly restored the HIND-FORE weight bearing difference to near to normal level compared to vehicle (n=8). Values are expressed mean+standard error (post hoc Tukey comparison, *p<0.05 and ** p<0.01 vs. vehicle, ##p<0.01 vs. sham).

FIGS. 12A-12B show the results of the withdrawal test for nociceptive behavior described in Example VI. Withdrawal threshold value in left and right hindlimbs of the MIA-induced LBP group decreased from 15 g to 2-3 g because of LBP development. Application of the mixed cell composition (n=7) significantly alleviated the sensitivity of hindlimbs to near normal level compared to vehicle (n=8). Values are expressed mean±standard error (post hoc Tukey comparison, **p<0.01 vs. vehicle, ##p<0.01 vs. sham).

FIG. 13 shows experimental design for the studies of mixed cells and individual cellular components of the mixed cells pain improvement effect described in Examples VII and VIII. Time schedules for the dynamic weight bearing test and withdrawal test in hindlimbs are illustrated. Injections of hChonJ-1 (2.25×10⁴, n=6); hChonJ-2 (4.5×10⁴, n=6); hChonJ #7-1 (0.75×10⁴, n=6); hChonJ #7-2 (3×10⁴ cells, n=6); the mixed cell composition (3×10⁴ cells, n=6), and vehicle (n=6) were performed 14 days after a surgery for creating the monosodium iodoacetate (MIA)-induced LBP model animals. Animals that underwent sham surgery were not injected with MIA or any test articles including vehicle and drug (n=6).

FIG. 14 shows the results of the dynamic weight bearing test for nociceptive behavior described in Example VII. Due to the development of LBP, the difference in weight bearing between the averaged hindlimbs and the averaged forelimbs (the HIND-FORE weight bearing difference) in the MIA-induced LBP group was reduced to 2-3%. Application of the mixed cell composition, but not of single components at various doses, significantly recovered the HIND-FORE weight bearing difference as compared to vehicle control. Values are expressed as mean±standard error (post hoc Tukey comparison, **p<0.01 vs. vehicle).

FIGS. 15A-15B show the results of the withdrawal test for nociceptive behavior described in Example VII. Due to the development of LBP, withdrawal threshold value in left hindlimbs (FIG. 15A) and right hindlimbs (FIG. 15B) of the MIA-induced group decreased to 3-4 g. Application of all single components did not significantly alleviate the sensitivity of hindlimbs compared to vehicle control. In contrast, application of the mixed cell composition significantly alleviated the sensitivity of hindlimbs compared to vehicle control. Values are expressed as mean±standard error (post hoc Tukey comparison, *, **p<0.01 vs. vehicle).

FIG. 16 shows the results of the dynamic weight bearing test for nociceptive behavior described in Example VIII. Like in Example VII, due to the development of LBP, the HIND-FORE weight bearing difference in the MIA-induced LBP group was reduced to 2-3%. Application of the mixed cell composition, but not of individual cellular components at various doses, significantly recovered the HIND-FORE weight bearing difference as compared to vehicle control. Values are expressed as mean±standard error (post hoc Tukey comparison, **p<0.01 vs. vehicle).

FIGS. 17A-17B show the results of the withdrawal test for nociceptive behavior described in Example VIII. Like in Example VII, due to the development of LBP, withdrawal threshold value in left hindlimbs (FIG. 17A) and right hindlimbs (FIG. 17B) of MIA-induced animals decreased to 3-4 g. The application of all single cellular components did not significantly alleviate the sensitivity of hindlimbs compared to vehicle control. In contrast, application of the mixed cell composition significantly alleviated the sensitivity of hindlimbs compared to vehicle control. Values are expressed as mean±standard error (post hoc Tukey comparison, *, **p<0.01 vs. vehicle).

FIG. 18 shows experimental design of the study of TG-C pain improvement dose effect described in Example IX. Time schedules for the dynamic weight bearing tests and withdrawal tests in hindlimbs are illustrated. Application of the mixed cell composition at different dosages (3×10³ total cells, 1×10⁴ total cells, and 3×10⁴ total cells, all n=8) or vehicle (n=8) was performed 14 days after creating the MIA-induced LBP model animals. Animals who underwent sham-surgery were not injected (n=8).

FIG. 19 shows the results of the weigh bearing test described in Example IX. Due to the development of LBP, the HIND-FORE weight bearing difference in the MIA-induced LBP group was reduced to 2-3%. However, application of the mixed cell composition at high and intermediate doses (3×10⁴ total cells and 1×10⁴ total cells; both n=8), but not at the low dose (3×10³ total cells; n=8) significantly alleviated the LBP-associated behavior as compared to vehicle control. Values are expressed as mean±standard error (post hoc Tukey comparison, ** p<0.01 vs. vehicle; #, ##p<0.05 or 0.01 vs sham).

FIGS. 20A-20B show the results of the withdrawal test for nociceptive behavior described in Example IX. Due to the development of LBP, withdrawal threshold value in left hindlimbs (FIG. 20A) and right hindlimbs (FIG. 20B) of the MIA-induced LBP animals decreased to 2-3 g. The application of the mixed cell composition at high and intermediate doses (3×10⁴ total cells and 1×10⁴ total cells; both n=8), but not at the low dose (3×10³ total cells, n=8), significantly alleviated the sensitivity of hindlimbs to near normal level compared to vehicle control (n=8). Values are expressed as mean±standard error (post hoc Tukey comparison, *, ** p<0.05 or 0.01 vs. vehicle; #, ##0.05 or 0.01 vs. sham).

FIG. 21 shows experimental design of the study TG-C pain improvement dose effect described in Example X. Time schedules for the dynamic weight bearing test and withdrawal test in hindlimbs are illustrated. Application of the mixed cell composition at different dosages (1×10⁴ total cells; 3×10⁴ total cells, and 5×10⁴ total cells, all n=8) or vehicle (n=8) was performed 14 days after creating the MIA-induced LBP model animals. Animals that underwent sham surgery were not injected (n=6).

FIG. 22 shows the results of the weight bearing test described in Example X. Like in Example IX, due to the development of LBP, the HIND-FORE weight bearing difference in the MIA-induced LBP animals was reduced to 3-4%. Application of TG-C at all doses significantly alleviated the LBP-associated behavior as compared to vehicle control (although some doses did not exert analgesic effects at 21 and 28 days of treatment in some of the animals). Values are expressed as mean±standard error (post hoc Tukey comparison, *, ** p<0.05 or 0.01 vs. vehicle).

FIGS. 23A-23B show the results of the withdrawal test for nociceptive behavior described in Example X. Due to the LBP development, withdrawal threshold value in left hindlimbs (FIG. 23A) and right hindlimbs (FIG. 23B) of the MIA-induced LBP animals decreased to 4 g. The application of the mixed cell composition at all doses (1.0×10⁴ total cells, 3.0×10⁴ total cells, and 5.0×10⁴ total cells; all groups n=8) significantly alleviated the sensitivity of hindlimbs to near normal level compared to vehicle control (n=8). Values are expressed as mean±standard error (post hoc Tukey comparison, *, ** p<0.05 or 0.01 vs. vehicle).

FIG. 24 shows experimental design of the study of TG-C pain reduction effect by reducing sensitivity of peripheral nerve system (via dorsal root ganglia “DRG” neurons) described in Example XI. Time schedules for the calcium imaging test are illustrated. Application of the mixed cell composition (3×10⁴, n=6) or vehicle (n=6) was performed 14 and 28 days after creating the MIA-induced LBP model animals. Animals that underwent sham surgery were not injected (n=6).

FIGS. 25A-25D show the results of the calcium imaging study at day 14 as described in Example XI. Dil+MIA+VEH group showed significant increase in sensitivity of DRG neurons as shown in the peak normalized ratio of 1 μM capsaicin- and 100 μM AITC-evoked calcium influx compared to unlabeled and Dil+SAL group. Importantly, Dil+MIA+mixed cells composition group significantly reduced the increase of calcium influx compared to Dil+MIA+VEH group and showed no difference of calcium influx compared to the unlabeled or Dil+SAL groups (##p<0.01 vs. Dil+MIA+VEH).

FIGS. 26A-26D show the results of the calcium imaging study at day 28 as described in Example XI. Dil+MIA+VEH group showed significant increase in the peak normalized ratio of 1 μM capsaicin- and 100 μM AITC-evoked calcium influx compared to unlabeled and DIL+SAL group. Importantly, Dil+MIA+mixed cells group significantly reduced the increase of calcium influx as compared to Dil+MIA+VEH group and showed no difference of calcium influx compared to the unlabeled and Dil+SAL groups (#p<0.05 vs. Dil+MIA+VEH).

### DETAILED DESCRIPTION

As used herein, the term “biologically active” in reference to a nucleic acid, protein, protein fragment or derivative thereof is defined as an ability of the nucleic acid or amino acid sequence to mimic a known biological function elicited by the wild type form of the nucleic acid or protein.

As used herein, the term “mammalian cells” in reference to transfected or transduced cells includes all types of mammalian cells, in particular human cells, including but not limited to connective tissue cells such as fibroblasts or chondrocytes, or stem cells, and in particular human embryonic kidney cells, and further in particular, human embryonic kidney 293 cells, or epithelial cells.

As used herein, the term “connective tissue” is any tissue that connects and supports other tissues or organs, and includes but is not limited to a ligament, a cartilage, a tendon, a bone, and a synovium of a mammalian host.

As used herein, the term “connective tissue cell” or “cell of a connective tissue” include cells that are found in the connective tissue, such as fibroblasts, cartilage cells (chondrocytes), and bone cells (osteoblasts/osteocytes), which secrete collagenous extracellular matrix, as well as fat cells (adipocytes) and smooth muscle cells. Preferably, the connective tissue cells are fibroblasts, chondrocytes, or bone cells. More preferably, the connective tissue cells are chondrocytes cells. It will be recognized that the invention can be practiced with a mixed culture of connective tissue cells, as well as cells of a single type. Preferably, the connective tissue cell does not cause a negative immune response when injected into the host organism. It is understood that allogeneic cells may be used in this regard, as well as autologous cells for cell-mediated gene therapy or somatic cell therapy.

As used herein, “connective tissue cell line” includes a plurality of connective tissue cells originating from a common parent cell.

As used herein, “hyaline cartilage” refers to the connective tissue covering the joint surface. By way of example only, hyaline cartilage includes, but is not limited to, articular cartilage, costal cartilage, and nose cartilage.

In particular, hyaline cartilage is known to be self-renewing, responds to alterations, and provides stable movement with less friction. Hyaline cartilage found even within the same joint or among joints varies in thickness, cell density, matrix composition and mechanical properties, yet retains the same general structure and function. Some of the functions of hyaline cartilage include surprising stiffness to compression, resilience, and exceptional ability to distribute weight loads, ability to minimize peak stress on subchondral bone, and great durability.

Grossly and histologically, hyaline cartilage appears as a slick, firm surface that resists deformation. The extracellular matrix of the cartilage comprises chondrocytes, but lacks blood vessels, lymphatic vessels or nerves. An elaborate, highly ordered structure that maintains interaction between chondrocytes and the matrix serves to maintain the structure and function of the hyaline cartilage, while maintaining a low level of metabolic activity. The reference O'Driscoll, J. Bone Joint Surg., 80A: 1795-1812, 1998 describes the structure and function of hyaline cartilage in detail, which is incorporated herein by reference in its entirety.

As used herein, “injectable” composition refers to a composition that excludes various three-dimensional scaffold, framework, mesh or felt structure, which may be made of any material or shape that allows cells to attach to it and allows cells to grow in more than one layer, and which structure is generally implanted, and not injected. In one embodiment, the injection method of the invention is typically carried out by a syringe. However, any mode of injecting the composition of interest may be used. For instance, catheters, sprayers, or temperature dependent polymer gels also may be used.

As used herein, “juvenile chondrocyte” refers to chondrocyte obtained from a human being who is less than two years old. Typically, chondrocyte is obtained from preferably the hyaline cartilage region of an extremity of the body, such as a finger, nose, ear lobe and so forth. Juvenile chondrocytes may be used as donor chondrocytes for allogeneic treatment of defected or injured intervertebral disc.

As used herein, the term “mammalian host” includes members of the animal kingdom including but not limited to human beings.

As used herein, “mixed cell” or a “mixture of cells” or “cell mixture” refers to the combination of a plurality of cells that include a first population of cells that are transfected or transduced with a gene or a nucleic acid sequence of interest and a second population of cells that are not transduced or transfected.

In some embodiments of the invention, mixed cells may refer to the combination of a plurality of cells that include cells that have been transfected or transduced with a gene or a nucleic acid sequence encoding a member of the TGF-β superfamily and cells that have not been transfected or transduced with a gene or a nucleic acid sequence encoding a member of the TGF-β superfamily. Typically, the ratio of cells that have not been transfected or transduced with a gene or a nucleic acid sequence encoding a member of TGF-β superfamily to cells that have been transfected or transduced with a gene or a nucleic acid sequence encoding a member of the TGF-β superfamily may be in the range of about 1-20 to 1. The range may include about 3-10 to 1. In particular, the range may be about 3 to 1. However, it is understood that the ratio of these cells is not necessarily limited to any particular range so long as the combination of these cells is effective to treat an injured intervertebral disc by slowing or retarding its degeneration.

The effective dose or therapeutically effective dose of the mixed cells for mammals including human may be in a rage from about 0.1×10⁶ to about 100×10⁶ cells. In some embodiments, the effective dose or therapeutically effective dose of the mixed cells for mammals including human may be in a rage from about 0.5×10⁶ to about 50×10⁶ cells.

In embodiments, the cells including transfected and non-transfected cells, are free of a scaffold or a three-dimensional support material that is pre-formed and shaped to accommodate the intended application, and the cells do not contain deposits of cells formed by, for example, bioprinting.

As used herein, “non-disc chondrocyte” refers to chondrocytes isolated from any part of the body except for intervertebral disc cartilage tissue. Non-disc chondrocytes of the present invention may be used for allogeneic transplantation or injection into a patient to treat defected or injured intervertebral disc.

As used herein, the term “patient” includes members of the animal kingdom including but not limited to human beings.

As used herein, the term “primed” cell refers to cells that have been activated or changed to express certain genes.

As used herein, low back pain refers to as pain and discomfort, localized below the costal margin and above the inferior gluteal folds, with or without leg pain. Non-specific low back pain is defined as low back pain not attributed to recognizable, known specific pathology and specific low back pain which has known pathomorphological cause.

As used herein, discogenic back pain refers to back pain or discomfort associated with intervertebral disc degeneration without herniation, anatomical deformity, or other alternate clear causes of pain and disability.

As used herein, discogenic low back pain refers to discogenic back pain localized below the costal margin and above the inferior gluteal folds, with or without leg pain.

As used herein, “slowing” or “prevention” of intervertebral disc degeneration refers to the retention of volume of intervertebral disc or height of the disc over time compared with the volume or height level that would normally be found at the site of injury leading to normal degeneration over a given time. This may mean a percentage increase of volume or height, such as about 10%, 20%, 30%, 40%, 50%, 60%, 70%, 80%, or 90% compared with the normal expected degeneration levels at a given time or may mean lessening of damage or depletion of volume or height of the intervertebral disc at the locus.

As used herein, the “transforming growth factor-β (TGF-β) superfamily” encompasses a group of structurally related proteins, which affect a wide range of differentiation processes during embryonic development. The family includes, Müllerian inhibiting substance (MIS), which is required for normal male sex development (Behringer, et al., Nature, 345:167, 1990), Drosophila decapentaplegic (DPP) gene product, which is required for dorsal-ventral axis formation and morphogenesis of the imaginal discs (Padgett, et al., Nature, 325:81-84, 1987), the Xenopus Vg-1 gene product, which localizes to the vegetal pole of eggs (Weeks, et al., Cell, 51:861-867, 1987), the activins (Mason, et al., Biochem, Biophys. Res. Commun., 135:957-964, 1986), which can induce the formation of mesoderm and anterior structures in Xenopus embryos (Thomsen, et al., Cell, 63:485, 1990), and the bone morphogenetic proteins (BMP's, such as BMP-2, 3, 4, 5, 6 and 7, osteogenin, OP-1) which can induce de novo cartilage and bone formation (Sampath, et al., J. Biol. Chem., 265:13198, 1990). The TGF-β gene products can influence a variety of differentiation processes, including adipogenesis, myogenesis, chondrogenesis, hematopoiesis, and epithelial cell differentiation (for a review, see Massague, Cell 49:437, 1987), which is incorporated herein by reference in its entirety.

The proteins of the TGF-β family are initially synthesized as a large precursor protein, which subsequently undergoes proteolytic cleavage at a cluster of basic residues approximately 110-140 amino acids from the C-terminus. The C-terminal regions of the proteins are all structurally related and the different family members can be classified into distinct subgroups based on the extent of their homology. Although the homologies within particular subgroups range from 70% to 90% amino acid sequence identity, the homologies between subgroups are significantly lower, generally ranging from only 20% to 50%. In each case, the active species appears to be a disulfide-linked dimer of C-terminal fragments. For most of the family members that have been studied, the homodimeric species has been found to be biologically active, but for other family members, like the inhibins (Ung, et al., Nature, 321:779, 1986) and the TGF-β's (Cheifetz, et al., Cell, 48:409, 1987), heterodimers have also been detected, and these appear to have different biological properties than the respective homodimers.

Members of the superfamily of TGF-β genes or proteins include TGF-β3, TGF-β2, TGF-β4 (chicken), TGF-β1, TGF-β5 (Xenopus), BMP-2, BMP-4, Drosophila DPP, BMP-5, BMP-6, Vgr1, OP-1/BMP-7, Drosophila 60A, GDF-1, Xenopus Vgf, BMP-3, Inhibin-βA, Inhibin-βB, Inhibin-α, and MIS. These genes and proteins are discussed in Massague, Ann. Rev. Biochem. 67:753-791, 1998, which is incorporated herein by reference in its entirety.

Preferably, the member of the superfamily of TGF-β genes and proteins is TGF-β1, TGF-β2, TGF-β3, BMP-2, BMP-3, BMP-4, BMP-5, BMP-6, or BMP-7.

Intervertebral Disc

The intervertebral discs make up one fourth of the spinal column's length. There are no discs between the Atlas (C1), Axis (C2), and Coccyx. Discs are not vascular and therefore depend on the end plates to diffuse needed nutrients. The cartilaginous layers of the end plates anchor the discs in place.

The intervertebral discs are fibrocartilaginous cushions serving as the spine's shock absorbing system, which protect the vertebrae, brain, and other structures (i.e. nerves). The discs allow some vertebral motion: extension and flexion. Individual disc movement is very limited—however considerable motion is possible when several discs combine forces.

Intervertebral discs are composed of an annulus fibrosus and a nucleus pulposus. The annulus fibrosus is a strong radial tire—like structure made up of lamellae; concentric sheets of collagen fibers connected to the vertebral end plates. The sheets are orientated at various angles. The annulus fibrosus encloses the nucleus pulposus.

Although both the annulus fibrosus and nucleus pulposus are composed of water, collagen, and proteoglycans (PGs), the amount of fluid (water and PGs) is greatest in the nucleus pulposus. PG molecules are important because they attract and retain water. The nucleus pulposus contains a hydrated gel—like matter that resists compression. The amount of water in the nucleus varies throughout the day depending on activity. As people age, the nucleus pulposus begins to dehydrate, which limits its ability to absorb shock. The annulus fibrosus gets weaker with age and begins to tear. While this may not cause pain in some people, in others one or both of these may cause chronic back pain.

Pain due to the inability of the dehydrating nucleus pulposus to absorb shock is called axial pain or disc space pain. One generally refers to the gradual dehydration of the nucleus pulposus as degenerative disc disease. When the annulus fibrosus tears due to an injury or the aging process, the nucleus pulposus can begin to extrude through the tear. This is called disc herniation. Near the posterior side of each disc, all along the spine, major spinal nerves extend out to different organs, tissues, extremities etc. It is very common for the herniated disc to press against these nerves (pinched nerve) causing radiating pain, numbness, tingling, and diminished strength and/or range of motion. In addition, the contact of the inner nuclear gel, which contains inflammatory proteins, with a nerve can also cause significant pain. Nerve-related pain is called radicular pain.

Intervertebral disc may be damaged by an injury or trauma or degenerated by aging. Damaged or degenerating intervertebral disc may be, but is not limited to, a thinning disc or a herniated disc. According to the embodiments of the present disclosure, the damaged or degenerating intervertebral disc may be restored structurally by increasing the height (or height index) and/or the volume of the damaged or degenerating disc (by, for example, increasing the content of water, collagen matrix, and/or proteoglycan in the damaged or degenerating disc) by administering an effective amount of a mixed cell composition as described herein to the damaged or degenerating intervertebral disc site. The administration route may be, but is not limited to, topical injection or transplantation of the mixed cell composition to the target damaged or degenerating disc site.

Herniated discs may be referred to by many names and these can mean different things to different medical professionals. A slipped disc, ruptured disc, or a bulging disc can all refer to the same medical condition. Protrusions of the disc into the adjacent vertebra are known as Schmorl's nodes.

Primed Cell Therapy

The present invention encompasses administering primed cells to an intervertebral disc region in a mammal to treat injured intervertebral disc by preventing or retarding degeneration of intervertebral disc. Primed cells are typically connective tissue cells, and include chondrocytes or fibroblasts.

The present invention encompasses administering primed cells to an intervertebral disc region in a mammal to prevent or treat back pain. According to an embodiment, the back pain could be caused by or associated with degeneration of intervertebral disc. Primed cells are typically connective tissue cells, and include chondrocytes or fibroblasts.

By way of example, when a population of primary chondrocytes are passaged about 3 or 4 times, their morphology typically changes to fibroblastic chondrocytes. As primary chondrocytes are passaged, they begin to lose some of their chondrocytic characteristics and begin to take on the characteristics of fibroblastic chondrocytes. When these fibroblastic chondrocytes are incubated or “primed” with a cytokine such as a protein from the TGF-β superfamily, the cells regain their chondrocytic characteristics, which include production of extracellular matrix such as collagens and proteoglycans.

Such primed cells include fibroblastic chondrocytes, which have been incubated with TGF-β1, and as a result have reverted to collagen producing chondrocytes. An advantage of using primed cells in retardation of intervertebral disc degeneration is the ease of creating useable chondrocytes for introduction into the intervertebral disc for production of collagen and otherwise maintenance of the cartilaginous matrix, and effectively relieving back pain.

The cells may include without limitation primary cells or cells which have undergone about one to twenty passages. The cells may be connective tissue cells. The cells may include cells that have undergone a morphogenic change, wherein the priming causes reversion to the characteristics of the original cell. The cells may include without limitation chondrocytes, fibroblasts, or fibroblastic chondrocytes. Priming may occur by incubating the cells for a period of at least 40 hours, or from 1 to 40 hours, from 2 to 30 hours, from 3 to 25 hours, from 4 to 20 hours, from 5 to 20, from 6 to 18 hours, 7 to 17 hours, 8 to 15 hours, or 9 to 14 hours, with a cytokine, and then optionally separating the cytokine from the cells and injecting the primed cells into a cartilaginous defect site of interest in order to regenerate cartilage, preferably hyaline cartilage. In one aspect, the cytokine may be a member of the superfamily of TGF-β. In particular, the cytokine may be TGF-β, and in particular, TGF-β1.

The cytokine may be present in the priming incubation mix in an amount to sufficiently “prime” the chondrocyte to be useful in the intervertebral treatment method. In this aspect, the priming incubation mix may contain at least about 1 ng/ml of the cytokine. In particular, the mix may contain from about 1 to 1000 ng/ml, from about 1 to 750 ng/ml, from about 1 to 500 ng/ml, from about 1 to 400 ng/ml, from about 1 to 300 ng/ml, from about 1 to 250 ng/ml, from about 1 to 200 ng/ml, from about 1 to 150 ng/ml, from about 1 to 100 ng/ml, from about 1 to 75 ng/ml, from about 1 to 50 ng/ml, from about 10 to 500 ng/ml, from about 10 to 400 ng/ml, from about 10 to 300 ng/ml, from about 10 to 250 ng/ml, from about 10 to 200 ng/ml, from about 10 to 150 ng/ml, from about 10 to 100 ng/ml, from about 10 to 75 ng/ml, from about 10 to 50 ng/ml, from about 15 to 500 ng/ml, from about 15 to 400 ng/ml, from about 15 to 300 ng/ml, from about 15 to 250 ng/ml, from about 15 to 200 ng/ml, from about 15 to 150 ng/ml, from about 15 to 100 ng/ml, from about 15 to 75 ng/ml, from about 15 to 50 ng/ml, from about 20 to 500 ng/ml, from about 20 to 400 ng/ml, from about 20 to 300 ng/ml, from about 20 to 250 ng/ml, from about 20 to 200 ng/ml, from about 20 to 150 ng/ml, from about 20 to 100 ng/ml, from about 20 to 75 ng/ml, from about 20 to 50 ng/ml, from about 25 to 500 ng/ml, from about 25 to 400 ng/ml, from about 25 to 300 ng/ml, from about 25 to 250 ng/ml, from about 25 to 200 ng/ml, from about 25 to 150 ng/ml, from about 25 to 100 ng/ml, from about 25 to 75 ng/ml, from about 25 to 50 ng/ml, from about 30 to 500 ng/ml, from about 30 to 400 ng/ml, from about 30 to 300 ng/ml, from about 30 to 250 ng/ml, from about 30 to 200 ng/ml, from about 30 to 150 ng/ml, from about 30 to 100 ng/ml, from about 30 to 75 ng/ml, from about 30 to 50 ng/ml, from about 35 to 500 ng/ml, from about 35 to 400 ng/ml, from about 35 to 300 ng/ml, from about 35 to 250 ng/ml, from about 35 to 200 ng/ml, from about 35 to 150 ng/ml, from about 35 to 100 ng/ml, from about 35 to 75 ng/ml, from about 35 to 50 ng/ml, from about 40 to 500 ng/ml, from about 40 to 400 ng/ml, from about 40 to 300 ng/ml, from about 40 to 250 ng/ml, from about 40 to 200 ng/ml, from about 40 to 150 ng/ml, from about 40 to 100 ng/ml, from about 40 to 75 ng/ml, or from about 40 to 50 ng/ml.

One method of practicing the invention may include incubating the cells with a cytokine for a certain length of time to create primed cells and optionally separating the cytokine from the cells, and injecting the primed cells into intervertebral disc or the site of interest near it. Alternatively, the cells may be incubated with the cytokine of interest for a time and the combination may be administered to the site of defect without separating out the cytokine.

It is to be understood that while it is possible that substances such as a scaffolding or a framework as well as various extraneous tissues may be implanted together in the primed cell therapy protocol of the present invention, it is also possible that such scaffolding or tissue not be included in the injection system of the invention. In a preferred embodiment, in the inventive somatic cell therapy, the invention is directed to a simple method of injecting a population of primed connective tissue cells to the intervertebral disc space.

It will be understood by the artisan of ordinary skill that the source of cells for treating a human patient may be the patient's own cells, but that allogeneic cells as well as xenogeneic cells may also be used without regard to the histocompatibility of the cells. Alternatively, in one embodiment of the invention, allogeneic cells may be used having matching histocompatibility to the mammalian host. To describe in further detail, the histocompatibility of the donor and the patient are determined so that histocompatible cells are administered to the mammalian host. Also, juvenile chondrocytes may also be used allogeneically without necessarily determining the histocompatibility of the donor and the patient.

Gene Delivery

In one aspect the present invention discloses ex vivo and in vivo techniques for delivery of a DNA sequence of interest to the connective tissue cells of the mammalian host. The ex vivo technique involves culture of target mammalian cells, in vitro transfection of the DNA sequence, DNA vector or other delivery vehicle of interest into the mammalian cells, followed by transplantation of the modified mammalian cells to the target area of the mammalian host, so as to effect in vivo expression of the gene product of interest.

It is to be understood that while it is possible that substances such as a scaffolding or a framework as well as various extraneous tissues may be implanted together in the protocol of the present invention, it is preferred that such scaffolding or tissue not be included in the injection system of the invention. In a one embodiment, the invention is directed to a simple method of injecting a TGF superfamily protein or a population of cultured, untransfected/untransduced connective tissue cells or transfected/transduced mammalian cells or a mixture thereof to the intervertebral disc space so that the exogenous TGF superfamily protein is expressed or is active in the space.

It will be understood by the artisan of ordinary skill that one source of cells for treating a human patient is the patient's own cells. Another source of cells includes allogeneic cells without regard to the histocompatibility of the cells to the patient sought to be treated.

More specifically, this method includes employing a gene product that is a member of the transforming growth factor β superfamily, or a biologically active derivative or fragment thereof, or a biologically active derivative or fragment thereof.

In another embodiment of this invention, a composition for parenteral administration to a patient in a therapeutically effective amount is provided that contains a TGF-β superfamily protein and a suitable pharmaceutical carrier.

Another embodiment of this invention provides a composition for parenteral administration to a patient in a prophylactically effective amount that includes a TGF-β superfamily protein and a suitable pharmaceutical carrier.

In therapeutic applications, the TGF-β protein may be formulated for localized administration. Techniques and formulations generally may be found in Remington's Pharmaceutical Sciences, Mack Publishing Co., Easton, Pa., latest edition. The active ingredient that is the TGF protein is generally combined with a carrier such as a diluent or excipient which may include fillers, extenders, binding, wetting agents, disintegrants, surface-active agents, biodegradable polymers, lubricants, preservatives, such as cryopreservatives, and others. A suitable carrier may be selected depending on the mode of administration and dosage form. The carrier may be or may contain a cryopreservation medium. The cryopreservation medium may contain about 10 to 20% w/w dimethyl sulfoxide (DMSO) and about 1 to 5 w/w % saccharose. Typical dosage forms include, powders, liquid preparations including suspensions, emulsions, solutions, granules, and capsules.

The TGF protein of the present invention may also be combined with a pharmaceutically acceptable carrier for administration to a subject. Examples of suitable pharmaceutical carriers are a variety of cationic lipids, including, but not limited to N-(1-2,3-dioleyloxy)propyl)-n,n,n-trimethylammonium chloride (DOTMA) and dioleoylphophotidyl ethanolamine (DOPE). Liposomes are also suitable carriers for the TGF protein molecules of the invention. Another suitable carrier is a slow-release gel or polymer comprising the TGF protein molecules.

The TGF beta protein may be mixed with an amount of a physiologically acceptable carrier or diluent, such as a saline solution or other suitable liquid. The TGF protein molecule may also be combined with other carrier means to protect the TGF protein and biologically active forms thereof from degradation until they reach their targets and/or facilitate movement of the TGF protein or biologically active form thereof across tissue barriers.

A further embodiment of this invention includes storing the cell prior to transferring the cells. It will be appreciated by those skilled in the art that the cells may be stored frozen in 10 percent DMSO in liquid nitrogen.

In the present application, a method is provided for regenerating or preventing degeneration of intervertebral disc, or for preventing or treating back pain, by injecting an appropriate mammalian cell that is transfected or transduced with a gene or a nucleic acid sequence encoding a member of the transforming growth factor-beta (TGF-β) superfamily, including, but not limited to, BMP-2 and TGF-β 1, 2, and 3.

In another embodiment of the present application, a method is provided for preventing or retarding degeneration of intervertebral disc, or for preventing or treating back pain, by injecting an appropriate connective tissue cell that is not transfected or transduced with a gene or a nucleic acid sequence encoding a member of the transforming growth factor-beta (TGF-β) superfamily or that is not transfected or transduced with any other gene. In another aspect, the invention is directed to treating injured or degenerated intervertebral disc by preventing or retarding degeneration of the intervertebral disc by using the above-described method.

In another embodiment of the present application, a method is provided for preventing or retarding degeneration of intervertebral disc, or for preventing or treating back pain, by injecting an appropriate mammalian cell that is transfected or transduced with a gene or a nucleic acid sequence encoding a member of the transforming growth factor-beta (TGF-β) superfamily. In another aspect, the invention is directed to treating injured or degenerated intervertebral disc by preventing or retarding degeneration of the intervertebral disc by using the above-described method.

In another embodiment of the present application, a method is provided for preventing or retarding degeneration of intervertebral disc, or for preventing or treating back pain, by injecting a combination of or a mixture of an appropriate mammalian cell that is transfected or transduced with a gene or a nucleic acid sequence encoding a member of the transforming growth factor-beta (TGF-β) superfamily and an appropriate connective tissue cell that is not transfected or transduced with a gene or a nucleic acid sequence encoding a member of the transforming growth factor-beta (TGF-β) superfamily or that is not transfected or transduced with any other gene. In another aspect, the invention is directed to treating injured or degenerated intervertebral disc by preventing or retarding degeneration of the intervertebral disc by using the above-described method.

In an embodiment of the invention, it is understood that the cells may be injected into the area in which degeneration of the intervertebral disc is to be sought to be prevented or retarded by using the cell above-described composition with or without scaffolding material or any other auxiliary material, such as extraneous cells or other biocompatible carriers. That is, the cells are free of a scaffold or a three-dimensional support material that is pre-formed and shaped to accommodate the intended application, and the mammalian cells do not contain deposits of cells formed by, for example, bioprinting. Thus, the modified cells alone, unmodified cells alone, or a mixture or combination thereof may be injected into the area in which the degeneration of the intervertebral disc is sought to be prevented or retarded, or the site of pain where the pain to be mitigated or alleviated.

The following examples are offered by way of illustration of the present invention, and not by way of limitation.

### EXAMPLES

### Example I—Materials and Methods

Plasmid Construction

The plasmid pMTMLVβ1 was generated by subcloning a 1.2-kb Bgl II fragment containing the TGF-β1 coding sequence and a growth hormone poly A site at the 3′ end into the Bam HI site of pMTMLV. pMTMLV vector was derived from the retroviral vector MFG by deleting entire gag and env sequences as well as some of iv packaging sequence.

Cell Culture and Transduction

The TGF-β cDNA cloned in retroviral vectors were individually transduced into 293 cells (293-TGF-β1). They were cultured in Dulbecco's Modified Eagle's Medium (GIBCO-BRL, Rockville, Md.) with 10% concentration of fetal bovine serum.

To select the cells transduced with the TGF-β1 coding sequence, neomycin (300 μg/ml) was added into the medium. The cells with TGF-β1 expression were sometimes stored in liquid nitrogen and cultured just before the injection.

Radiographic Analysis of Disc Height

Radiographs were taken after administration of ketamine hydrochloride (25 mg/kg) and Rompun (1 mg/kg) at various week intervals after the puncture. Extreme care was taken to maintain a consistent level of anesthesia during radiography of each animal and at each time to obtain a similar degree of muscle relaxation, which may affect the disc height. Therefore, the preoperative radiograph was always used as a baseline measurement. Efforts were also made to keep the spine in a slightly flexed position. To decrease the error from axial rotation of the spine and beam divergence, radiographs were repeated at least twice on each animal in the lateral decubitus position, with the beam centered at 4 cm from the rabbit iliac crest. Radiographs were digitally scanned and digitally stored using an Image Capture software.

Image Analysis

Using digitized radiographs, measurements, including the vertebral body height and intervertebral disc (IVD) height, were analyzed using the public domain image analysis. The data were transported to Excel software, and the IVD height was expressed as the disc height index (DHI) using the method of Lu et al. “Effects of chondroitinase ABC and chymopapain on spinal motion segment biomechanics. An in vivo biomechanical, radiologic, and histologic canine study”, Spine 1997; 22:1828-34. Average IVD height (DHI) was calculated by averaging the measurements obtained from the anterior, middle, and posterior portions of the IVD and dividing that by the average of adjacent vertebral body heights. Changes in the DHI of injected discs were expressed as percent DHI and normalized to the measured preoperative IVD height (percent DHI=postoperative DHI/preoperative DHI×100). The within-subject standard deviation (Sw) was calculated using the equation:

√(Σ(x₁−x₂)²/2n)

Where X₁ is the first measurement value, X₂ is the second measurement value, and n=450. The percent coefficient of variance (percent CV) was calculated as (Sw/means of all measurements×100). The intra-observer error of DHI measurements was estimated to be minimal (Sw: 0.001800316; percent CV: 3.13). The interobserver error was also reported to be small (Sw: 0.003227; percent CV: 9.6)

MRI Assessments

MRI examinations were performed on all rabbits in the study using a 0.3-T imager (Airis II, version 4.0 A; Hitachi Medical System America, Inc.) with a quadrature extremity coil receiver. After sacrifice, the spinal columns with surrounding soft tissue were isolated and subjected to MRI analysis. T2-weighted sections in the sagittal plane were obtained in the following settings: fast spin echo sequence with TR (time to repetition) of 4000 milliseconds and TE (time to echo) of 120 milliseconds; 256 (h)×128 (v) matrix; field of view of 260; and 4 excitations. The section thickness was 2 mm with a 0-mm gap. A blinded observer using the modified Thompson classification based on changes in the degree and area of signal intensity from grade 1 to 4 (1=normal, 2=minimal decrease of signal intensity but obvious narrowing of high signal area, 3=moderate decrease of signal intensity, and 4=severe decrease of signal intensity) evaluated MRIs. The intraobserver and interobserver reliability correlation coefficients of MRI grading based on 2 evaluations were excellent (K=0.98, 0.90, respectively), as determined by the Cohen kappa correlation coefficient.

### Example II—Experimental Methods and Results

Preventing Degeneration of Injured Intervertebral Disc

New Zealand white male rabbits were used. An open surgical technique was used. Three intervertebral levels in the lumbar spine: L2-3, L3-4, L4-5 were experimentally treated or observed as a control in each animal. Treatments were assigned to levels in a balanced manner with multiple sites/discs per rabbit observed. Within subject design, pre-post-surgery comparisons, change across disc levels were used as controls.

### Example III

Preventing Degeneration of Injured Intervertebral Disc Using Untransduced Chondrocyte Alone, TGF-B1-Producing 293 Cells Alone, or with Mixed-Cells (Human Chondrocytes and TGF-B1-Producing 293 Cells) Injection in Rabbits

All of the chondrocytes used in Examples I-V are non-disc chondrocytes and are juvenile chondrocytes, obtained from the hyaline cartilage portion of a finger of a less than two year old child.

Needle puncture was produced in the intervertebral discs of the lumbar spine. After this needle puncture, TGF-β1-producing 293 cells, primary untransduced human chondrocytes, mixture of TGF-β1-producing 293 cells and primary untransduced human chondrocytes, primed untransduced human chondrocytes or carrier/media are injected. Several controls are used. Experimental conditions are listed in Table I.

TABLE I

| Surgical | |
|---|---|
| Preparation | Injection Treatment |
| Needle puncture | TGF-β1-producing 293 cells |
| | (~5 × 10⁶cells) |
| Needle Puncture | Mixed: TGF-β1-producing 293 cells |
| | Primary untransduced human chondrocytes |
| | (~3 to 1 ratio, 5 × 10⁶) |
| Needle Puncture | Primary untransduced human chondrocytes |
| | (~5 × 10⁶) |
| Needle Puncture | Primed untransduced human chondrocytes |
| | (~5 × 10⁶) |
| Needle puncture | DMEM |
| Needle puncture | Needle puncture only-no injection |
| No puncture | No puncture no treatment control |

Briefly, a needle puncture injury is produced in the intervertebral discs of the lumbar spine of rabbit or a pig. After this needle puncture, rabbits are left to heal for 4 weeks. Then in a second surgical procedure, experimental treatment composition, which includes TGF-β1-producing 293 cells and/or primary untransduced human chondrocytes (˜5×10⁵) is injected or control conditions observed (Table I).

After endotrachial intubation and general anesthesia is achieved such as by administration of ketamine hydrochloride and ROMPTJN®, the animal is placed in supine position. Lactated ringers are used at about (5 ml/kg/hr). The area of incision is shaved and prepped and draped in the usual sterile fashion with alternating betadine scrubs and alcohol wipes (>three times). Bland ophthalmic ointment is placed on the eyes. A left retroperitoneal approach is used to expose the right anterior aspect of the disc from L2-L5 (the rabbit has 6 to 7 lumbar vertebra). Various preparation schemes are used and treatment schema is applied to each disc level. For ‘Needle Puncture’ preparation of the disc, a 18-gauge needle is used to place a puncture in the disc at the depth of 5 mm (Aoki et al., “Nerve fiber ingrowth into scar tissue formed following nucleus pulposus extrusion in the rabbit annular-puncture disc degeneration model: effects of depth of puncture.” Spine. 2006; 31(21):E774-80). After puncture, the test materials listed in Table I are injected. Treatment composition is applied to any one of L1-2, L2-3, L3-4, L4-5 region of each rabbit.

Monthly radiographs are used to monitor any disc changes. Animals are sacrificed at 2, 8, and 24 weeks after surgery.

Radiographs/MRI. Healing is indicated by a detectable radiographic change of increased disc height from same disc at baseline (pre op) compared to disc at other disc levels. Other discs are compared before and after needle puncture only, and disc before and after no needle puncture yielding an index of normal degeneration over time.

Retro-Transcription PCR. Retro-transcription PCR is performed to assay relative quantity of surviving transfected chondrocytes.

Histology. Also histology is used to confirm characterization of Type I and II Collagen and the gross appearance and evaluation of de novo chondrocytes.

Western Blot analysis and or ELISA. Quantitative expression of collagen type I and type II, and proteoglycan concentration, Smads 2/3, Sox-9. Additionally, ELISA is used to evaluate TGFβ-1, BMP2, BMP7, GDF5 and other related growth factors where there are available antibodies.

Apoptosis is examined in the other tissue structures of the intervertebral disc via observing the expression of Capase-3.

### Example IV

Results

The results are as shown in the Figures and the description of the Figures of the present application. Punctured intervertebral disc treated with untransduced chondrocytes alone, transduced 293 cells alone, primed chondrocyte alone or a mixture of transduced 293 cells and untransduced chondrocytes, show beneficial effects in preventing or retarding disc degeneration compared with vehicle control.

### Example IV-1—Mixed-Cell (Transduced 293 Cells and Untransduced Chondrocytes) Treatment of Punctured Intervertebral Disc in Rabbit

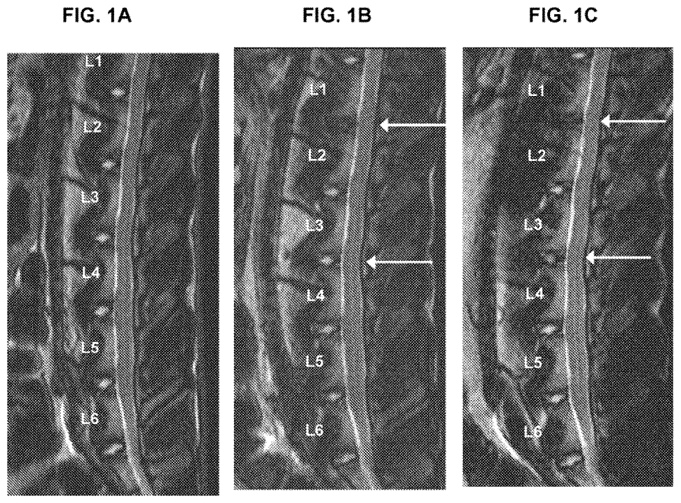

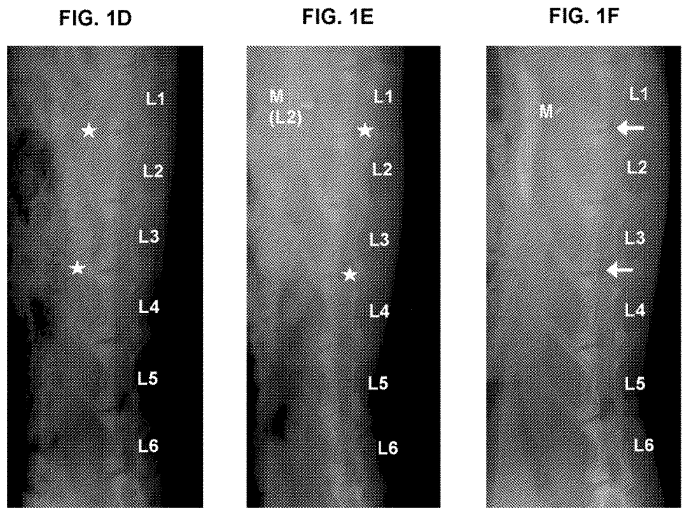

Mixed cell treatment has an intervertebral anti-degenerating effects when tested on rabbits. The effect is seen in a variety of experiments in FIGS. 1-4. FIGS. 1A-1F show slowing, retardation, or prevention of degeneration of injured disc. (A) shows MRI radiograph of rabbit spine pre-surgery; (B) shows MRI radiograph of a rabbit spine four (4) weeks after surgery in which (i) the disc at L1/2 was injured and TGF-β1-producing 293 cells were injected, (ii) no puncture and no treatment is seen at spine locus L2/3, and (iii) disc at L3/4 was injured and mixture of TGF-β1-producing 293 cells and untransduced human chondrocytes in 1:3 ratio were injected; arrows point to L1/2 and L3/4 disc region. (C) shows MRI radiograph of a rabbit spine eight (8) weeks after surgery in which (i) the disc at L1/2 was injured and TGF-β1-producing 293 cells were injected, (ii) no puncture and no treatment control at spine locus L2/3, and (iii) disc at L3/4 was injured and mixture of TGF-β1-producing 293 cells and untransduced human chondrocytes in 1:3 ratio were injected; arrows point to L1/2 and L3/4 disc region. (D) shows X-ray radiograph of the rabbit described in (A) above, which is used to obtain a disc height index of the intervertebral disc to measure its morphology, its level of degeneration or regeneration. (E) shows X-ray radiograph of the rabbit described in (B) above, which is used to obtain a disc height index of the intervertebral disc. (F) shows X-ray radiograph of the rabbit described in (C) above, which is used to obtain a disc height index of the intervertebral disc.

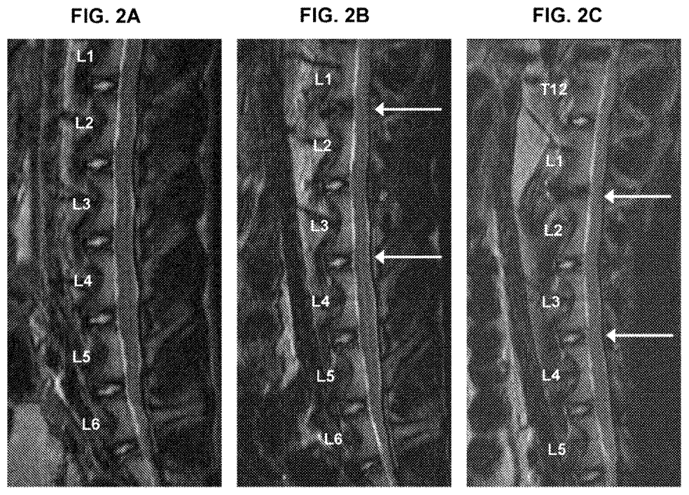

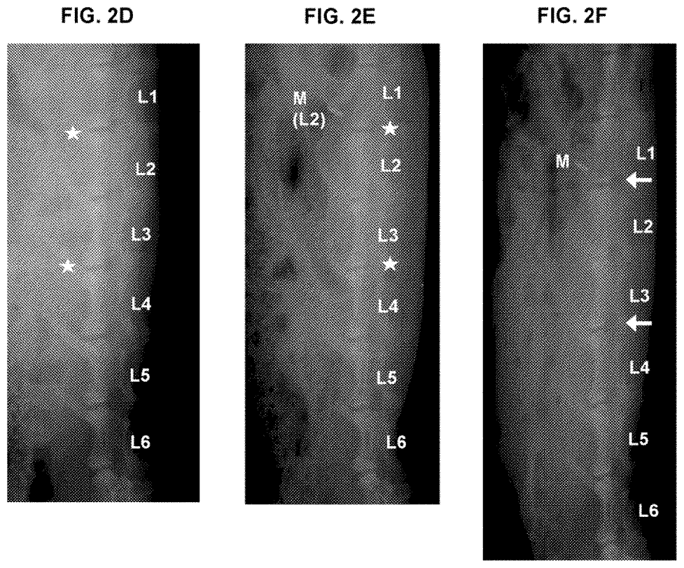

FIGS. 2A-2F show slowing, retardation, or prevention of degeneration of injured disc. (A) shows MRI radiograph of rabbit spine pre-surgery; (B) shows MRI radiograph of a rabbit spine four (4) weeks after surgery in which (i) the disc at L1/2 was injured and TGF-β1-producing 293 cells were injected, (ii) no puncture and no treatment control at spine locus L2/3, and (iii) disc at L3/4 was injured and mixture of TGF-β1-producing 293 cells and untransduced human chondrocytes in 1:3 ratio were injected; arrows point to L1/2 and L3/4 disc region. (C) shows MRI radiograph of a rabbit spine eight (8) weeks after surgery in which (i) the disc at L1/2 was injured and TGF-β1-producing 293 cells were injected, (ii) no puncture and no treatment is seen at spine locus L2/3, and (iii) disc at L3/4 was injured and mixture of TGF-β1-producing 293 cells and untransduced human chondrocytes in 1:3 ratio were injected; arrows point to L1/2 and L3/4 disc region. (D) shows X-ray radiograph of the rabbit described in (A) above, which is used to obtain a disc height index of the intervertebral disc to measure its morphology, its level of degeneration or regeneration. (E) shows X-ray radiograph of the rabbit described in (B) above, which is used to obtain a disc height index of the intervertebral disc. (F) shows X-ray radiograph of the rabbit described in (C) above, which is used to obtain a disc height index of the intervertebral disc.

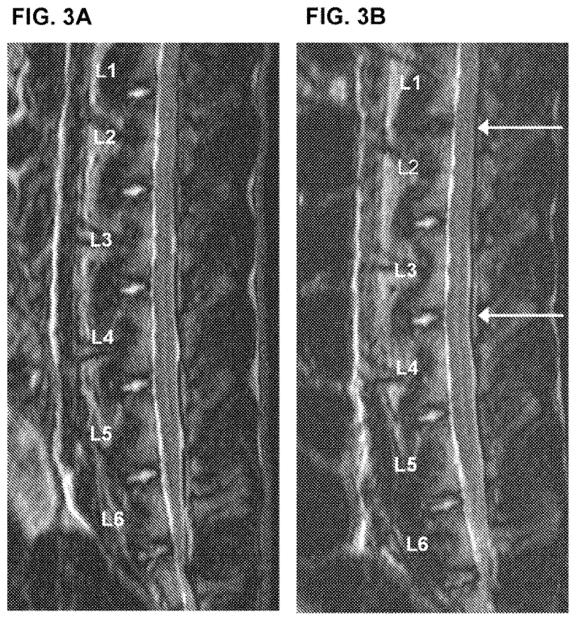

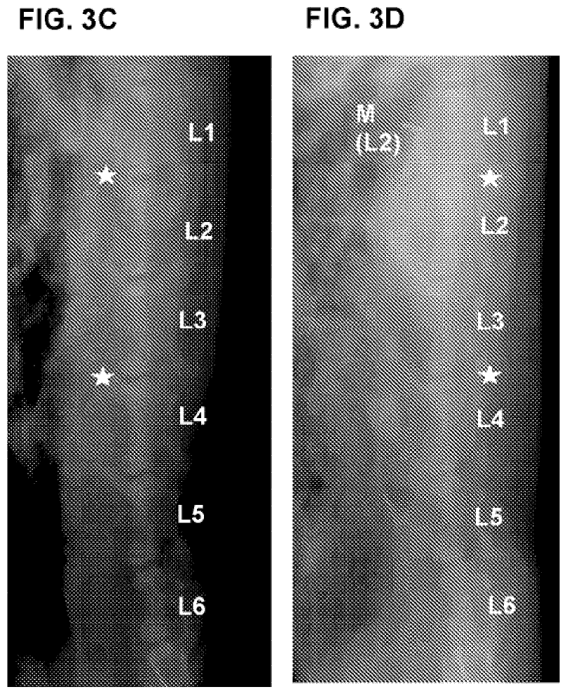

FIGS. 3A-3D show a slowing, retardation or prevention of degeneration of injured disc. (A) shows MRI radiograph of rabbit spine pre-surgery; (B) shows MRI radiograph of a rabbit spine four (4) weeks after surgery in which (i) the disc at L1/2 was injured and TGF-β1-producing 293 cells were injected, (ii) no puncture and no treatment control at spine locus L2/3, and (iii) disc at L3/4 was injured and mixture of TGF-β1-producing 293 cells and untransduced human chondrocytes in 1:3 ratio were injected; arrows point to L1/2 and L3/4 disc region. (C) shows X-ray radiograph of the rabbit described in (A) above, which is used to obtain a disc height index of the intervertebral disc to measure its morphology, its level of degeneration or regeneration. (D) shows X-ray radiograph of the rabbit described in (B) above, which is used to obtain a disc height index of the intervertebral disc.

### Example IV-2—Transduced 293 Cell Treatment of Punctured Intervertebral Disc in Rabbit

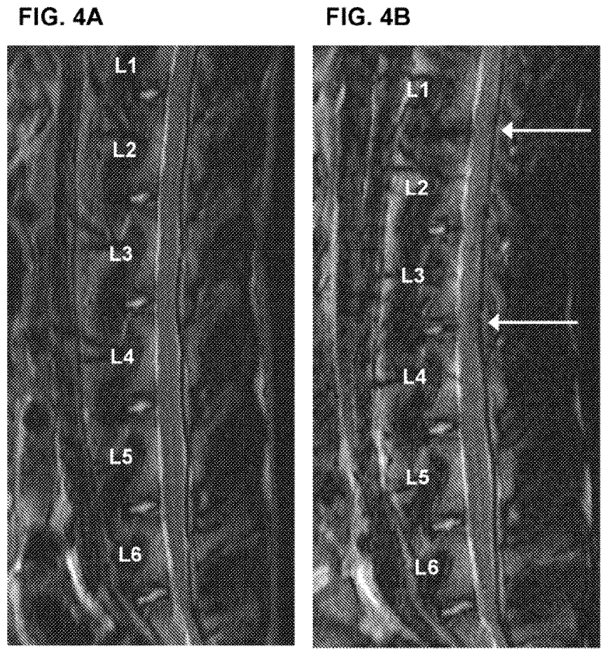

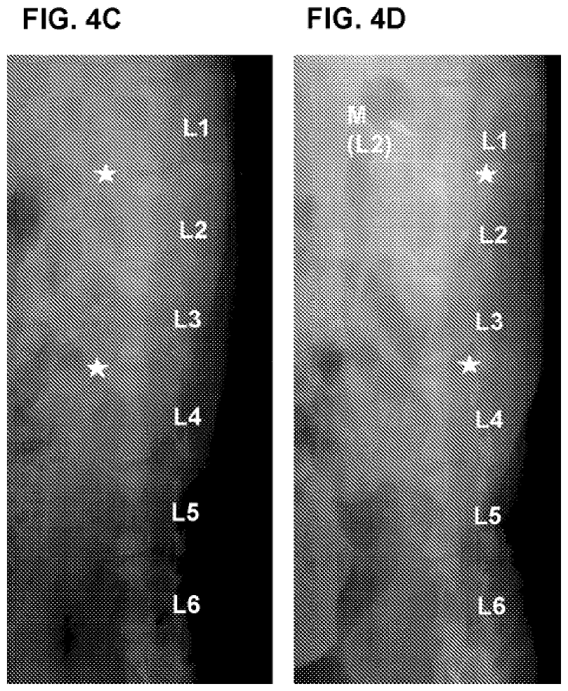

TGF-β1-producing 293 cells treatment has an intervertebral anti-degenerating effect. The effect is seen in FIGS. 4A-4D, which show slowing, retardation, or prevention of degeneration of injured disc. (A) shows MRI radiograph of rabbit spine pre-surgery; (B) shows MRI radiograph of a rabbit spine four (4) weeks after surgery in which (i) the disc at L1/2 was injured and mixture of TGF-β1-producing 293 cells and untransduced human chondrocytes in 1:3 ratio were injected, (ii) no puncture and no treatment control at spine locus L2/3, and (iii) disc at L3/4 was injured and TGF-β1-producing 293 cells were injected; arrows point to L1/2 and L3/4 disc regions. (C) shows X-ray radiograph of the rabbit described in (A) above, which is used to obtain a disc height index of the intervertebral disc to measure its morphology, its level of degeneration or regeneration. (D) shows X-ray radiograph of the rabbit described in (B) above, which is used to obtain a disc height index of the intervertebral disc.

### Example IV-3—Transduced 293 Cell Treatment and Mixed-Cell Treatment of Punctured Intervertebral Disc in Rabbit

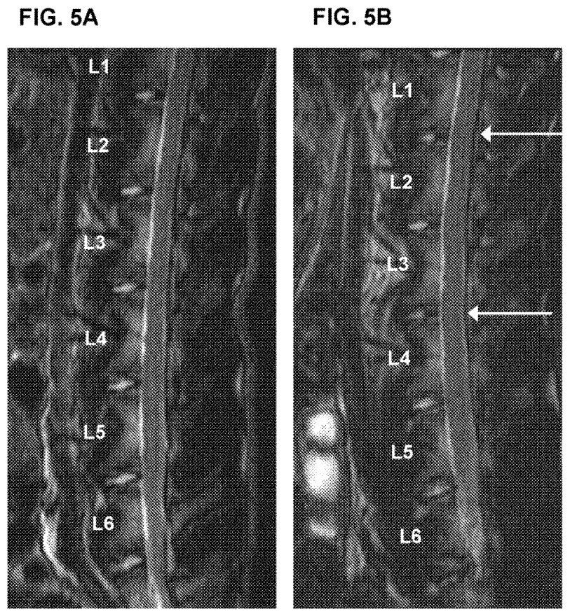

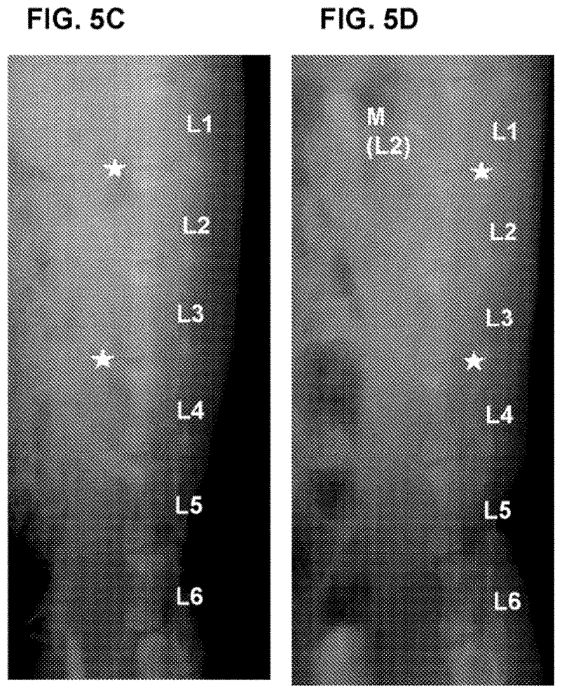

TGF-β1-producing 293 cell treatment and mixed cell treatments have an intervertebral anti-degenerating effect. The effect is seen in FIGS. 5A-5D, which show a slowing, retardation or prevention of degeneration of injured disc. (A) shows MRI radiograph of rabbit spine pre-surgery; (B) shows MRI radiograph of a rabbit spine four (4) weeks after surgery in which (i) the disc at L1/2 was injured and mixture of TGF-β1-producing 293 cells and untransduced human chondrocytes in 1:3 ratio were injected, (ii) no puncture and no treatment control at spine locus L2/3, and (iii) disc at L3/4 was injured and TGF-β1-producing 293 cells were injected; arrows point to L1/2 and L3/4 disc regions. (C) shows X-ray radiograph of the rabbit described in (A) above, which is used to obtain a disc height index of the intervertebral disc to measure its morphology, its level of degeneration or regeneration. (D) shows X-ray radiograph of the rabbit described in (B) above, which is used to obtain a disc height index of the intervertebral disc.

### Example IV-4—Untransduced Chondrocyte Treatment of Punctured Intervertebral Disc in Rabbit

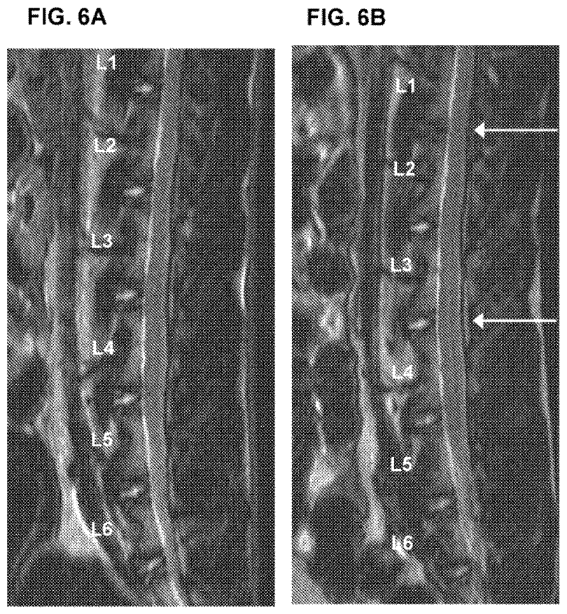

Untransduced chondrocyte treatment has an intervertebral anti-degenerating effect. The effect is seen in a variety of experiments in FIGS. 6-8. FIGS. 6A-6D show a slowing, retardation or prevention of degeneration of injured disc. (A) shows MRI radiograph of rabbit spine pre-surgery; (B) shows MRI radiograph of a rabbit spine four (4) weeks after surgery in which (i) the disc at L1/2 was injured and cell culture media DMEM was injected, (ii) no puncture and no treatment control at spine locus L2/3, and (iii) disc at L3/4 was injured and untransduced chondrocytes were injected; arrows point to L1/2 and L3/4 disc regions. (C) shows X-ray radiograph of the rabbit described in (A) above, which is used to obtain a disc height index of the intervertebral disc to measure its morphology, its level of degeneration or regeneration. (D) shows X-ray radiograph of the rabbit described in (B) above, which is used to obtain a disc height index of the intervertebral disc.

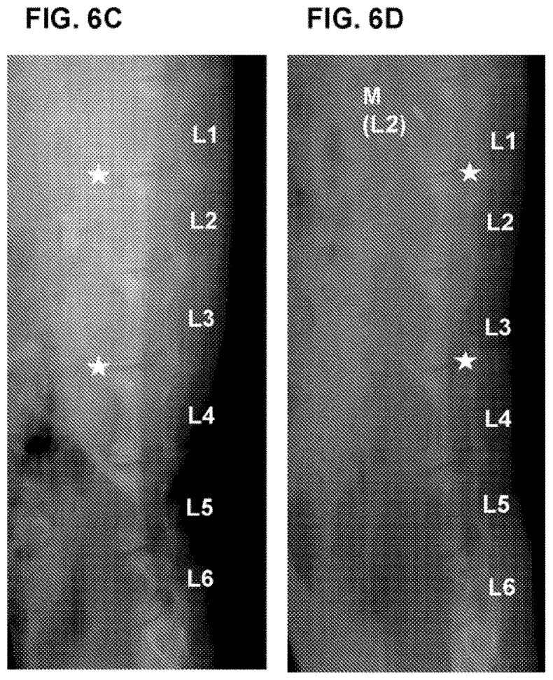

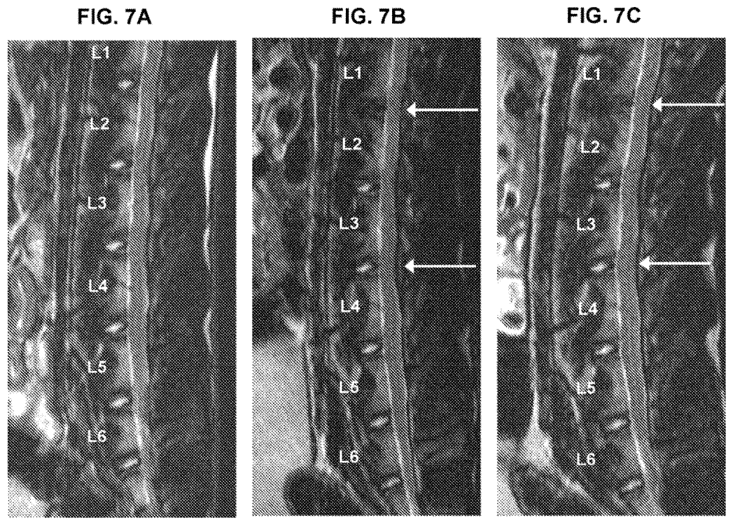

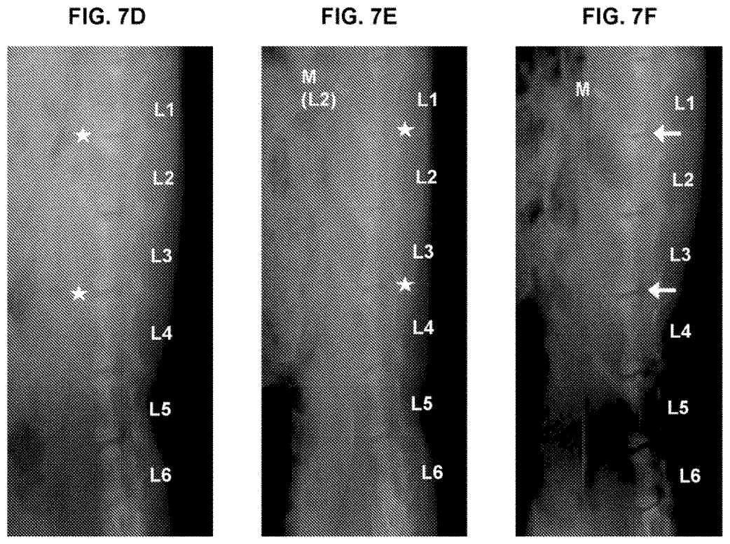

FIGS. 7A-7F show slowing, retardation, or prevention of degeneration of injured disc. (A) shows MRI radiograph of rabbit spine pre-surgery; (B) shows MRI radiograph of a rabbit spine four (4) weeks after surgery in which (i) the disc at L1/2 was injured and cell culture DMEM was injected, (ii) no puncture and no treatment control at spine locus L2/3, and (iii) disc at L3/4 was injured and untransduced chondrocytes were injected; arrows point to L1/2 and L3/4 disc regions. (C) shows MRI radiograph of a rabbit spine eight (8) weeks after surgery in which (i) the disc at L1/2 was injured and cell culture media DMEM was injected, (ii) no puncture and no treatment control at spine locus L2/3, and (iii) disc at L3/4 was injured and untransduced chondrocytes were injected; arrows point to L1/2 and L3/4 disc regions. (D) shows X-ray radiograph of the rabbit described in (A) above, which is used to obtain a disc height index of the intervertebral disc to measure its morphology, its level of degeneration or regeneration. (E) shows X-ray radiograph of the rabbit described in (B) above, which is used to obtain a disc height index of the intervertebral disc. (F) shows X-ray radiograph of the rabbit described in (C) above, which is used to obtain a disc height index of the intervertebral disc.

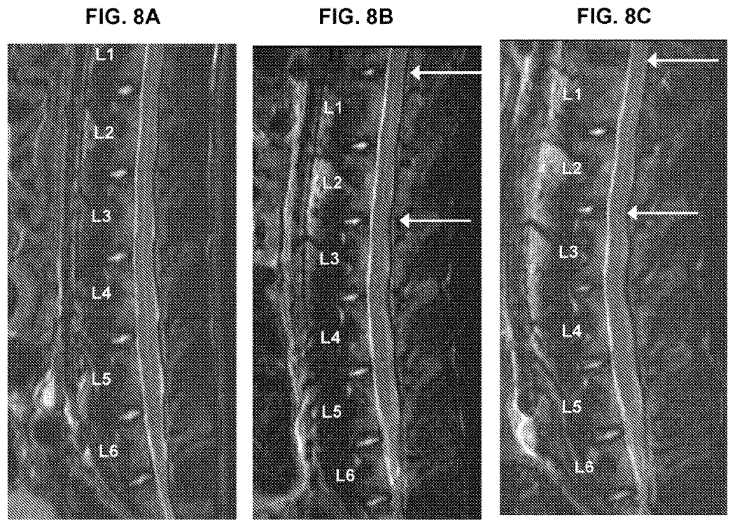

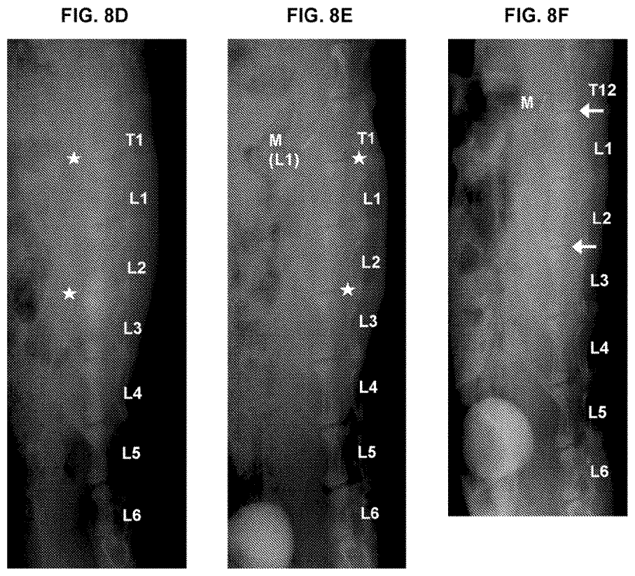

FIGS. 8A-8F show slowing, retardation, or prevention of degeneration of injured disc. (A) shows MRI radiograph of rabbit spine pre-surgery; (B) shows MRI radiograph of a rabbit spine four (4) weeks after surgery in which (i) the disc at T12/L1 was injured by needle puncture and no injection, (ii) no puncture and no treatment control at spine locus L1/2, and (iii) disc at L2/3 was injured and untransduced chondrocytes were injected; arrows point to T12/L1 and L2/3 disc regions. (C) shows MRI radiograph of a rabbit spine eight (8) weeks after surgery in which (i) the disc at T12/L1 was injured by needle puncture and no injection, (ii) no puncture and no treatment control at spine locus L1/2, and (iii) disc at L2/3 was injured and untransduced chondrocytes were injected; arrows point to T12/L1 and L2/3 disc regions. (D) shows X-ray radiograph of the rabbit described in (A) above, which is used to obtain a disc height index of the intervertebral disc to measure its morphology, its level of degeneration or regeneration. (E) shows X-ray radiograph of the rabbit described in (B) above, which is used to obtain a disc height index of the intervertebral disc. (F) shows X-ray radiograph of the rabbit described in (C) above, which is used to obtain a disc height index of the intervertebral disc.

### Example IV-5—Untransduced Primed Chondrocyte Treatment of Punctured Intervertebral Disc in Rabbit

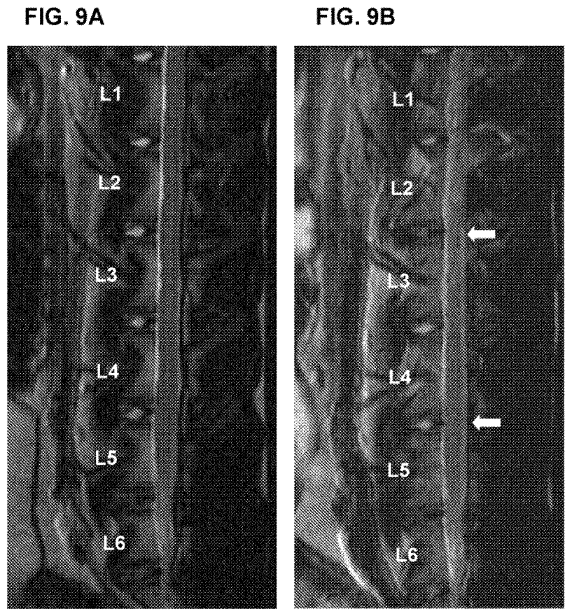

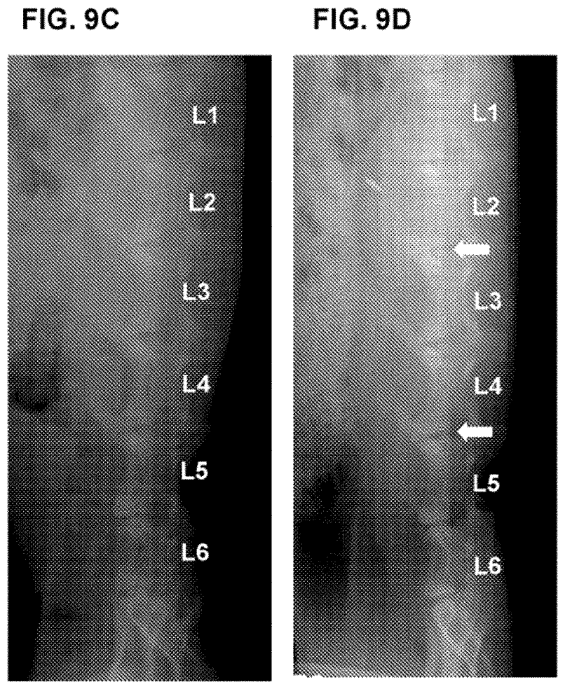

Primed chondrocyte treatment has an intervertebral anti-degenerating effect. The effect is seen in FIGS. 9A-9D, which show slowing, retardation, or prevention of degeneration of injured disc. (A) shows MRI radiograph of rabbit spine pre-surgery; (B) shows MRI radiograph of a rabbit spine eight (8) weeks after surgery in which (i) the disc at L2/3 was injured and cell culture media DMEM was injected, (ii) no puncture and no treatment control at spine locus L3/4, and (iii) disc at L4/5 was injured and primed chondrocytes were injected; arrows point to L2/3 and L4/5 disc regions. (C) shows X-ray radiograph of the rabbit described in (A) above, which is used to obtain a disc height index of the intervertebral disc to measure its morphology, its level of degeneration or regeneration. (D) shows X-ray radiograph of the rabbit described in (B) above, which is used to obtain a disc height index of the intervertebral disc.

### Example V

Source of Human Chondrocytes

Primary human chondrocytes were grown from cartilage tissue obtained from the surgical excision of a polydactyly finger from a one-year-old female human donor. The polydactyl tissue was harvested in a surgical room. The following procedure for chondrocyte isolation was performed in a biosafety cabinet. The plastic bottle containing the cartilage tissue was swiped with alcohol and the cartilage tissue was washed with sterile PBS (1×) using a pipette. A collagenase solution was prepared by dissolving 7 mg of collagenase (Gibco BRL) in 10 mL of DMEM (containing 10% FBS) and filtering through a 0.2 μm syringe filter (Corning). The washed cartilage tissue was treated with the collagenase solution for 17 to 18 hrs in a 37° C. shaker incubator. On the following day, the bottle was sanitized with alcohol. The collagenase treated material was pipetted up and down several times to separate loose cells from the tissue mass. After pipetting, the supernatant was filtered through 70 μm nylon cell strainer (Falcon). Collagenase treated tissue which had lost its integrity (e.g., loose cells) was able to pass through the filter. The cell filtrate was collected in a 50 mL tube (Falcon) and then centrifuged at 1,500 rpm for 5 minutes. Two thirds of the supernatant was discarded and the pellet washed with 10 ml of sterile PBS (1×). The resuspended cells were again centrifuged at 1,500 rpm for 5 minutes and, after removal of two-thirds of the supernatant, washed with 10 ml of sterile PBS (1×). The cells were again centrifuged at 1,500 rpm for 5 minutes and then resuspended in DMEM (containing 10% FBS). The resuspended cells were then transferred to four uncoated 25 cm² flasks and cultured for four days at 37° C. with 5% CO₂. The cells were then transferred into two uncoated 185 cm² flasks. The cells were cultured for two weeks and then collected, washed and resuspended in a cryopreservative media of DMEM, FBS and DMSO in a 5:4:1 ratio. The cells were aliquotted in to cryovials containing 1 mL of cell suspension at 4×10⁵ cells/mL. The cells were held in vapor phase liquid nitrogen storage.

### Example VI—Efficacy of Mixed-Cell Treatment for Alleviating Discogenic Low Back Pain in Monosodium Iodoacetate-Induced Low Back Pain Animal Model

Lumbar spine is a critical region for LBP. In the MIA-induced LBP animal model, MIA is injected into the lumbar 4/5 (L4/5) and 5/6 (L5/6) IVD of rats inducing IVD inflammation and associated pain. The experiments described herein investigated whether treatment with a mix of human allogeneic chondrocytes and irradiated GP2-293 cells expressing TGF-β1 (mixed cell treatment or mixed cells) can relieve pain by measuring two types behavior: dynamic weight bearing and withdrawal threshold in hindlimbs of MIA-induced discogenic LBP rats.

Animals

Male Sprague-Dawley rats (230-250 g, n=25) were used for generating the MIA-induced animal model of discogenic LBP. Food and water were available ad libitum.

Generating MLA-Induced Animal Model of Discogenic Low Back Pain

Rats were anesthetized with 5 mg/kg alfaxalone and 0.25 mg/kg medetomidine in oxygen, and a midline ventral longitudinal incision was made. Two-level lumbar disc 4/5 (L4/5) and 5/6 (L5/6) were exposed under a microscope. For establishing MIA-induced animal model of discogenic low back pain (the rats of this model are referred to herein as the MIA-induced LBP model animals), a dose of MIA (1 mg/μl, 2 μl; dissolved in normal 0.9% saline) was individually injected into these lumbar discs with a Hamilton syringe (26s G needle). Muscles and skin margins were sutured with 6-0 and 5-0 silk. Sham control group underwent the same surgical procedure, except that intradiscal MIA injection following the disc exposure was not performed.

Treatment

Mixed cell composition contained 1.0×10⁵ total cells comprising a mixture of 7.5×10⁴ human allogeneic chondrocytes (hChonJ) and 2.5×10⁴ irradiated GP2-293 cells expressing TGF-β1 (hChonJ #7), corresponding to the 3 to 1 of hChonJ to hChonJ #7. A DMSO-containing cryopreservation medium (CRYOSTOR® CS-10, BioLifeSolution, WA, USA) was used as a vehicle control. Treatment or vehicle were administered to intradiscal space of L4/5 and L5/6 of rats in 2 μL total volume by injection with a Hamilton syringe (31 G needle). Treatment and excipient compositions are listed in Table II.

TABLE II
Treatment Materials

| | |
|---|---|
| Name | hChonJ |
| Ingredient | Human allogeneic chondrocytes |
| Dose | 1.5 × 10⁷ cells/mL in 1.65 mL |
| Storage | LN2 Vapor Phase |
| Name | hChonJ#7 |
| Ingredient | Irradiated GP2-293 cells expressing TGF-β1 |
| Dose | 1.5 × 10⁷ cells/mL in 0.65 mL |
| Storage | LN2 Vapor Phase |

Excipient

| | |
|---|---|
| Name | CS10 (CRYOSTOR ® CS10) |
| Manufacturer | BioLifeSolutions |
| Size | 100 mL |
| Storage | 2-8° C. |

Treatment Preparation and Administration

### Cell Thawing:

1) Frozen cells were thawed in a 37° C. water bath.
2) The thawed cells were diluted using pre-warmed DMEM media to a total volume of 10 mL.
3) The cell suspension was centrifuged at 480×g for 5 minutes at 4° C.
4) DMEM was removed and cell pellet was resuspended in PBS (hChonJ cells: 3 mL; hChonJ #7: 1 mL).

### Cell Counting:

1) Trypan blue 90 μL and cell suspension 10 μL were transferred E-tube (Dilution factor: 10), and mixed by pipetting.
2) 10 μL of Trypan blue and cell suspension mixture was loaded to C-chip (Disposable hemocytometer).
3) Viable (seen as bright cells) and non-viable cells (stained blue) were counted in 4 squares of 1 mm² area of the C-chip.
4) Cell number was calculated as follows: Total cell number=Counted cell number in 4 squares of 1 mm² area/4×Dilution factor×PBS volume for resuspension.

### Mixed Cell Composition Preparation:

1) Mixed cells (hChonJ about 7.5×10⁶ cells and hChonJb #7 about 2.5×10⁶ cells) were placed in a conical tube and centrifuged at 480×g for 5 minutes at 4° C.
2) Supernatant was removed and CS-10 was added to make a final volume of 200 μL.

### Administration:

A 31 G needle was applied to the Hamilton syringe and the syringe was loaded to administer 2 μL of treatment or vehicle per injection site.

Dynamic Weight Bearing

The weight load carried by the four limbs of freely walking rats bearing a weight-bearing device was measured as follows. The bottom of the device was equipped with a load cell sensor (CB1-K2, DACELL, Cheongju, Korea), and output signals were fed to a digital amplifier (DN-AM 300, DACELL, Cheongju, Korea) for appropriate amplification and filtering. The signal was digitized via an analogue-digital converter (1716, DACELL, Cheongju, Korea) and plotted as a time-weight curve on a personal computer. The test was repeated three or four times to obtain at least eight to ten time-weight curves for a given limb. The weight bearing value of individual animal was normalized into a percentage of body weight. The HIND-FORE weight bearing difference was used for analysis.

Withdrawal Threshold in Hindlimbs

To measure the mechanical threshold for hindlimb withdrawal, a series of von Frey filaments (0.41-15.10 g, Stoelting, USA) were applied. Under a transparent plastic dome (28×28×10 cm) on a metal mesh floor, a von Frey filament was applied to the plantar surface of the right or left hindlimb. A 50% withdrawal threshold was determined using an up-down procedure, and stimuli were presented at intervals of several seconds. A brisk foot withdrawal to the von Frey filament application was regarded as a positive response. Interpolation of the 50% threshold was performed using the Dixon method.

Experimental Design

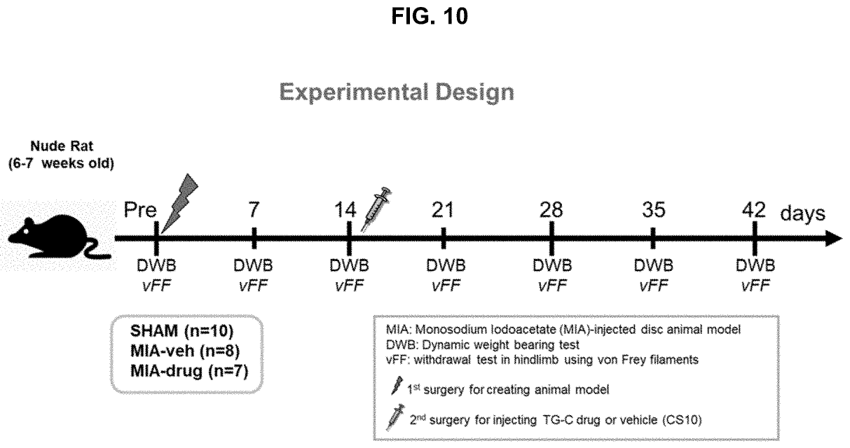

The experimental design of this study is illustrated in FIG. 10. The MIA-induced animal model of discogenic LBP was established as described above. Animals were divided into three groups: a group that underwent sham surgery (SHAM; n=10); a group that underwent surgery with MIA injection and a second surgery with vehicle injection (MIA-veh; n=8); a group that underwent surgery with MIA injection and a second surgery with injection of the mixed cells (MIA-drug; n=7).

Animals underwent surgery for generating LBP model inducted by injecting with MIA or sham (no surgery and MIA injection). The second surgery for intradiscal injection of the mixed cells, single components or vehicle was performed on MIA-injected animals 14 days after the first surgery. Dynamic weight bearing test and withdrawal threshold test in left and right hindlimbs were used for repetitive LBP-associated behavior testing (7 trials in total) were performed according to the following schedule: just before the first surgery, 7 days post-first surgery, 14 days post-first surgery (which is just before the second surgery) and at 7 days intervals thereafter up to day 42 post-first surgery.

Statistical Analysis

Data is expressed as means with standard error and evaluated using SPSS software version 28 (IBM corp., USA). Parametric or non-parametric statistics analysis of data was used depending on the pass of normal distribution testing. Dynamic weight load bearing and withdrawal threshold were analyzed using a two-way repeated measures ANOVA with a post hoc Tukey test. Differences between experimental conditions are considered statistically significant when p<0.05.

Results

The mixed cell treatment significantly alleviates MIA-induced LBP.

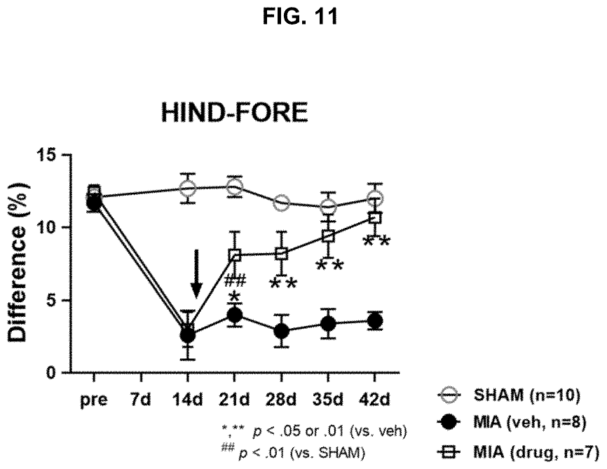

As shown in FIG. 11, the LBP-associated behavior was strongly expressed 14 days after the intradiscal injection of MIA. The HIND-FORE weight bearing difference was reduced from 12% to 3%; withdrawal threshold in hindlimb was reduced from 15 g to 4 g. Intradiscal injection of the mixed cells significantly alleviated the LBP-associated behavior.

Post hoc analysis indicates that mixed cell-treated animals demonstrated significant recovery of dynamic weight bearing (increased HIND-FORE weight bearing difference) at 21 to 42 days post treatment compared to vehicle-treated animals (21 days, p<0.05; 28-42 days, p<0.01). See FIG. 11.

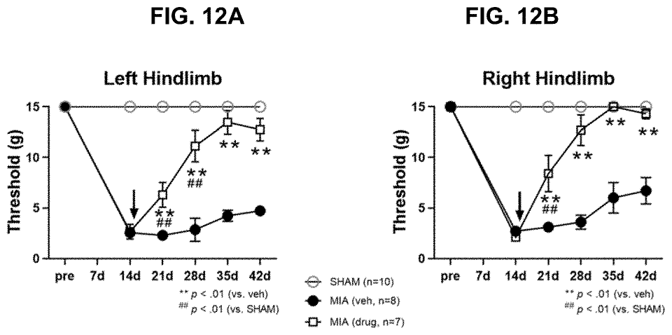

In parallel, the mixed cell treatment significantly increased the withdrawal threshold in left hindlimbs (FIG. 12A) and right hindlimbs (FIG. 12B) of the MIA-induced LBP model animals at 21 to 42 days post treatment injection compared to vehicle-treated animals (all days, p<0.01).

The mixed cell treatment demonstrates consistent suppressive effect on LBP as demonstrated by the dynamic weight bearing changes and withdrawal threshold to hindlimbs.

### Example VII—Efficacy of Mixed-Cell and Single Component Treatment for Alleviating Discogenic Low Back Pain in Monosodium Iodoacetate-Induced Discogenic Low Back Pain Model

Animals

Male Sprague-Dawley rats (270-290 g, n=42) were used for creating an animal model of discogenic LBP. Food and water were available ad libitum.

MIA-Induced Animal Model of Discogenic Low Back Pain

MIA-induced Animal Model of Discogenic Low Back Pain was generated as described in Example VI.

Treatment

The mixed cell treatment contained 3.0×10⁴ total cells comprising a mixture of human allogeneic chondrocytes (hChonJ) and irradiated GP2-293 cells expressing TGF-β1 (hChonJ #7) at the ratio of 3 to 1. Single component treatments were as follows: hChonJ-1 containing 2.25×10⁴ human allogeneic chondrocytes; hChonJ-2 containing 4.5×10⁴ human allogeneic chondrocytes; hChonJb #7-1 containing 0.75×10⁴ irradiated GP2-293 cells expressing TGF-β1; and hChonJb #7-2 containing 3×10⁴ irradiated GP2-293 cells expressing TGF-β1. CRYOSTOR® CS10, BioLifeSolution, WA, USA, was used as a vehicle control. Treatments or vehicle were administered to intradiscal space of L4/5 and L5/6 of rats in 2 μL total volume by injection with a Hamilton syringe (31 G needle). Treatment and excipient compositions are listed in Table II.

TABLE II
Treatment Materials

| | |
|---|---|
| Name | hChonJ |
| Ingredient | Human allogeneic chondrocytes |
| Dose | 1.5 × 10⁷ cells/mL in 1.65 mL |
| Storage | LN2 Vapor Phase |
| Name | hChonJ#7 |
| Ingredient | Irradiated GP2-293 cells expressing TGF-β1 |
| Dose | 1.5 × 10⁷ cells/mL in 0.65 mL |
| Storage | LN2 Vapor Phase |

Excipient

| | |
|---|---|
| Name | CS10 (CRYOSTOR ® CS10) |
| Manufacturer | BioLifeSolutions |
| Size | 100 mL |
| Storage | 2-8° C. |

Treatment Preparation and Administration

Cell thawing and counting were performed as described in Example VI.

### Mixed Cell Composition Preparation:

1) Mixed cells (3×10⁴ total cells per disc, containing 2.25×10⁴ hChonJ cells and 0.75×10⁴ hChonJb #7 cells), hChonJ-1 (2.25×10⁴ cells per disc), hChonJ-2 (4.5×10⁴ cells per disc), hChonJb #7-1 (0.75×10⁴ cells per disc), and hChonJb #7-2 (3×10⁴ cells per disc) were placed in each conical tube and centrifuged at 480×g for 5 minutes at 4° C.
2) Supernatant was removed and CS-10 was added to make a final volume of 2 μL per disc.

Administration was performed as described in Example VI.

Dynamic Weight Bearing

The weight load carried by the four limbs of freely walking rats bearing a weight-bearing device was measured as follows. The bottom of the device was equipped with a load cell sensor (CB1-K2, DACELL, Cheongju, Korea), and output signals were fed to a digital amplifier (DN-AM 300, DACELL, Cheongju, Korea) for appropriate amplification and filtering. The signal was digitized via an analogue-digital converter (1716, DACELL, Cheongju, Korea) and plotted as a time-weight curve on a personal computer. The test was repeated three or four times to obtain at least eight to ten time-weight curves for a given limb. The weight bearing value of individual animal was normalized into a percentage of body weight. The HIND-FORE weight bearing difference was used for analysis.

Withdrawal Threshold in Hindlimbs

To measure the mechanical threshold for hindlimb withdrawal, a series of von Frey filaments (0.41-15.10 g, Stoelting, USA) were applied. Under a transparent plastic dome (28×28×10 cm) on a metal mesh floor, a von Frey filament was applied to the plantar surface of the right or left hindlimb. A 50% withdrawal threshold was determined using an up-down procedure, and stimuli were presented at intervals of several seconds. A brisk foot withdrawal to the von Frey filament application was regarded as a positive response. Interpolation of the 50% threshold was performed according to the method of Dixon.

Experimental Design

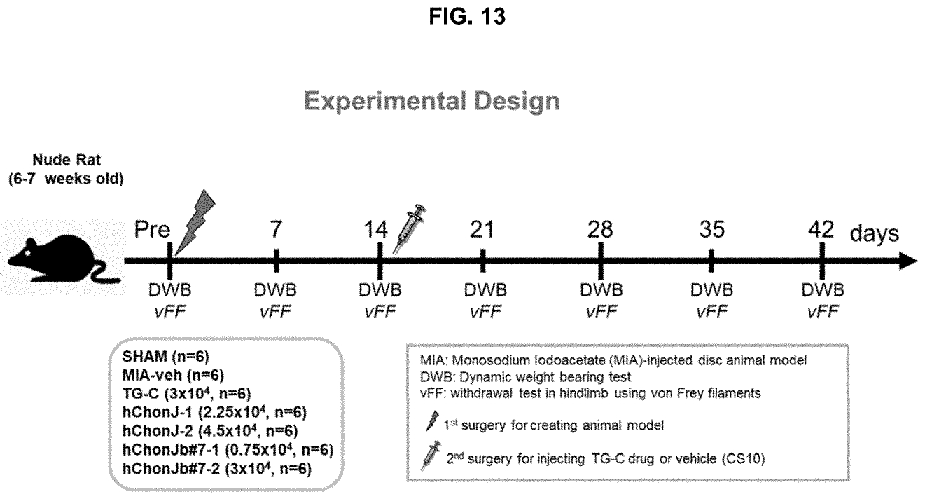

The experimental design of this study is illustrated in FIG. 13. The MIA-induced LBP model animals were generated as follows. Animals were divided into seven groups: a group that underwent sham surgery (SHAM; n=6); a group that underwent surgery with MIA and vehicle injection (MIA-veh; n=6); a group that underwent surgery and MIA injection and injection with one of the following: mixed cells (3×10⁴ total cells) (TG-C; n=6), MIA-hChonJ-1 (2.25×10⁴ cells, n=6); MIA-hChonJ-2 (4.5×10⁴ cells, n=6); MIA-hChonJ #7-1 (0.75×10⁴ cells, n=6); and MIA-hChonJ #7-2 (3×10⁴ cells, n=6). Animals that underwent sham surgery were not injected.

Two consecutive surgical procedures were performed on all animals: first surgery for model induction by MIA injection or sham surgery; and second surgery, 14 days after first surgery, for intradiscal injection of mixed cells, single components or vehicle. Dynamic weight bearing test and withdrawal threshold test in left and right hindlimbs were used for repetitive LBP-associated behavior testing (7 trials in total) were performed according to the following schedule: just before the first surgery, 7 days post-first surgery, 14 days post-first surgery (which as just before the second surgery) and at 7 day intervals thereafter up to day 42 post-first surgery.

Statistical Analysis

Data is expressed as means with standard error and evaluated using SPSS software version 28 (IBM corp., USA). Parametric or non-parametric statistics analysis of data was used depending on the pass of normal distribution testing. Dynamic weight load bearing and withdrawal threshold were analyzed using a two-way repeated measures ANOVA with a post hoc Tukey test. Differences between experimental conditions are considered statistically significant when p<0.05.

Results

Mixed cell treatment, but not single component treatments, significantly alleviates MIA-induced LBP.

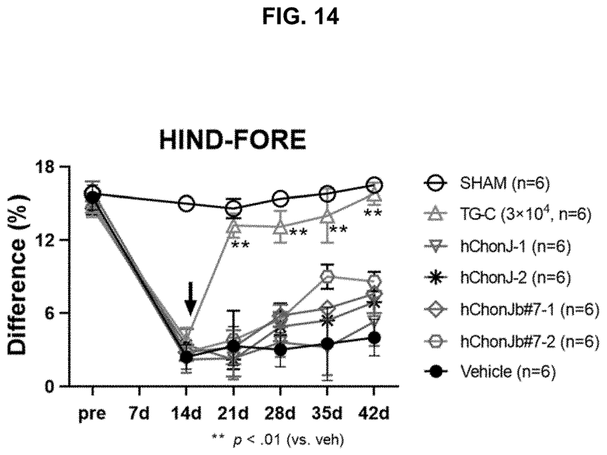

As shown in FIG. 14, the LBP-associated behavior was strongly expressed 14 days after the intradiscal injection of MIA. The HIND-FORE weight bearing difference was reduced from 15% to 2%; withdrawal threshold in hindlimb was reduced from 15 g to 4 g. All single components at various dosages (hChonJ-1, 2.25×10⁴ cells; hChonJ-2, 4.5×10⁴ cells; hChonJ #7-1, 0.75×10⁴ cells; hChonJ #7-2, 3×10⁴ cells; all n=6) did not significantly alleviate the LBP-associated behavior as compared to vehicle control, whereas treatment with mixed cells (n=6) significantly alleviated the LBP-associated behavior.

Post hoc analysis indicates that mixed cell-treated animals, but not single component-treated animals, demonstrated significant recovery of dynamic weight bearing (increased HIND-FORE weight bearing difference) at 21 to 42 days post treatment injection compared to vehicle-treated animals (21-42 days, all p<0.01). See FIG. 14.

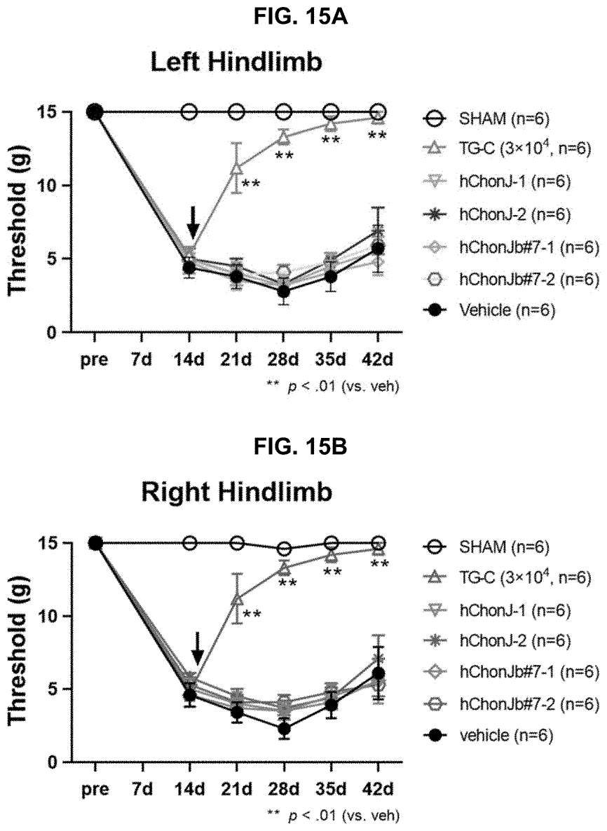

In parallel, mixed cell treatment, but not single component treatments, significantly increased the withdrawal threshold in left hindlimbs (FIG. 15A) and right hindlimbs (FIG. 15B) of MIA-induced model animals at 21 to 42 days post treatment injection compared to vehicle-treated animals (21-42 days, all p<0.01).

### Example VIII—Efficacy of Mixed-Cell and Single Component Treatment for Alleviating Discogenic Low Back Pain in Monosodium Iodoacetate-Induced Discogenic Low Back Pain Model

All procedures were performed as described in Example VII.

Treatment materials are shown in Table III.

TABLE III
Treatment Materials

| | |
|---|---|
| Name | hChonJ |
| Ingredient | Human allogeneic chondrocytes |
| Dose | 1.5 × 10⁷ cells/mL in 1.65 mL |
| Storage | LN2 Vapor Phase |

0

| | |
|---|---|
| Name | hChonJ#7 |
| Ingredient | Irradiated GP2-293 cells expressing TGF-β1 |
| Dose | 1.5 × 10⁷ cells/mL in 0.65 mL |
| Storage | LN2 Vapor Phase |

Excipient

| | |
|---|---|
| Name | CS10 (CRYOSTOR ® CS10) |
| Manufacturer | BioLifeSolutions |
| Size | 100 mL |
| Storage | 2-8° C. |

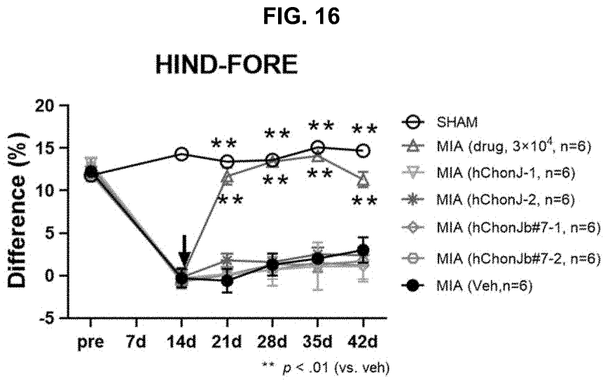

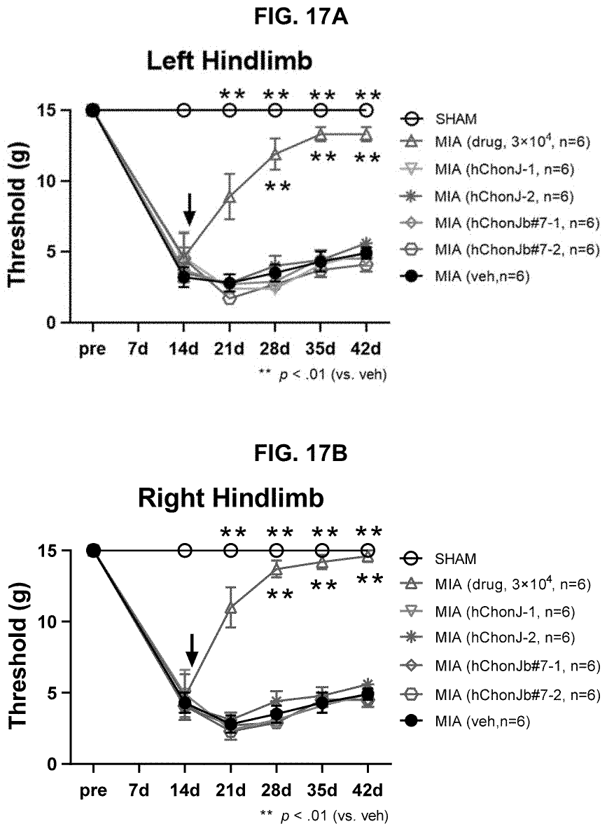

The results of the weight bearing test are shown in FIG. 16. The results of the withdrawal test are shown in FIGS. 17A-17B.

Examples VII and VIII demonstrate that mixed cell treatment (3×10⁴ total cells) has consistent suppressive effect on LBP as measured by the dynamic weight load bearing and withdrawal threshold to hindlimbs, whereas single component treatments at various dosages do not. It appears that a single component treatment does not provide for optimal condition for creating an analgesic effect.

### Example IX—Dose-Dependent Efficacy of Mixed-Cell Treatment for Alleviating Discogenic Pain in Monosodium Iodoacetate-Induced Discogenic Low Back Pain Model

Animals

Male Sprague-Dawley rats (240-270 g, n=42) were used for creating an animal model of discogenic LBP. Food and water were available ad libitum.

MIA-Induced Animal Model of Discogenic Low Back Pain

The model animals were generated as described in Example VI.

Treatment

Mixed cell compositions contained 3.0×10³ total cells, 1.0×10⁴ total cells, or 3.0×10⁴ total cells comprising a mixture of human allogeneic chondrocytes (hChonJ) and irradiated GP2-293 cells expressing TGF-β1 (hChonJ #7) at the ratio of 3 to 1. CRYOSTOR® CS-10, BioLifeSolution, WA, USA, was used as a vehicle control. Treatments or vehicle were administered to intradiscal space of rats in 2 μL total volume by injection with a Hamilton syringe (31 G needle). Treatment and excipient compositions are listed in Table IV.

TABLE IV
Treatment Materials

| | |
|---|---|
| Name | hChonJ |
| Ingredient | Human allogeneic chondrocytes |
| Dose | 1.5 × 10⁷ cells/mL in 1.65 mL |
| Storage | LN2 Vapor Phase |
| Name | hChonJ#7 |
| Ingredient | Irradiated GP2-293 cells expressing TGF-β1 |
| Dose | 1.5 × 10⁷ cells/mL in 0.65 mL |
| Storage | LN2 Vapor Phase |

Excipient

| | |
|---|---|
| Name | CS-10 (CRYOSTOR ® CS-10) |
| Manufacturer | BioLifeSolutions |
| Size | 100 mL |
| Storage | 2-8° C. |

Treatment Preparation and Administration Cell Thawing and cell counting were performed as described in Example VI.

### Mixed Cell Preparation:

1) hChonJ (1.69×10⁵, 5.63×10⁵, or 1.69×10⁶ cells) and hChonJb #7 (5.63×10⁴, 1.88×10⁵, or 5.63×10⁵ cells) were placed in each conical tube and centrifuged at 480×g for 5 minutes at 4° C.
2) Supernatant was removed and CS-10 was added to make a final volume of 150 pt. Administration was performed as described in Example VI.

Dynamic Weight Bearing

The test was performed as described in Example VI. The test was repeated three or four times to obtain at least eight to ten time-weight curves for a given limb.

Withdrawal Threshold in Hindlimbs

The test was performed as described in Example VI.

Experimental Design

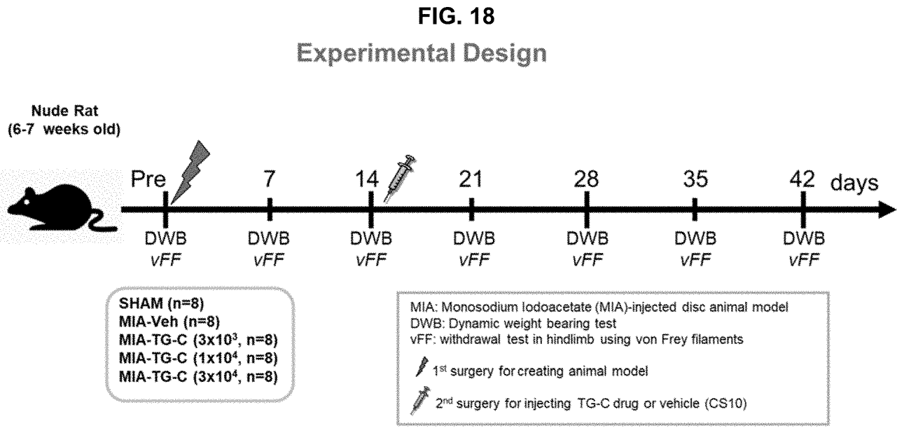

The experimental design is illustrated in FIG. 18. MIA-induced animal model of discogenic LBP was established as described in Example VI.

Animals were divided into five groups: sham surgery (SHAM; n=10); surgery with MIA and vehicle injection (MIA-veh; n=8); and surgery and injection with MIA and mixed cells at three doses (MIA-TG-C): 3.0×10³ total cells, 1.0×10⁴ total cells, and 3.0×10⁴ total cells (all MIA-TG-C groups n=8).

Two consecutive surgical procedures were performed on all animals: first surgery for model induction by MIA injection or sham surgery; and second surgery, 14 days after first surgery, for intradiscal injection of mixed cells or vehicle. Dynamic weight bearing and withdrawal threshold in left and right hindlimbs were used for repetitive LBP-associated behavior testing (7 trials in total), were performed according to the following schedule: just before the first surgery, 7 days post-first surgery, 14 days post-first surgery (which as just before the second surgery) and at 7 day intervals thereafter up to day 42 post-first surgery.

Statistical Analysis

Statistical analysis was performed as described in Example VI.

Results

Mixed cell treatment significantly alleviates MIA-induced LBP at all dosages.

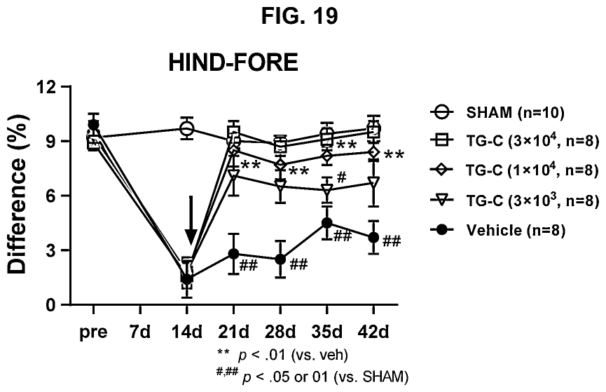

The results of the weigh bearing test are shown in FIG. 19. The LBP-associated behavior was strongly expressed 14 days after the intradiscal injection of MIA. The HIND-FORE weigh bearing difference was reduced from 10% to 2%; withdrawal threshold in hindlimb was reduced from 15 g to 2 g. Intradiscal injections of mixed cells at high and intermediate doses (3×10⁴ total cells and 1×10⁴ total cells; all n=8), but not the low dosage (3×10³ total cells; n=8) significantly alleviated the LBP-associated behavior as compared to vehicle control.

Post hoc analysis indicates that MIA-injected animals treated with intermediate and high doses of mixed cells demonstrated significant recovery of dynamic weight bearing (increased HIND-FORE difference) compared to vehicle-treated animals 21 to 42 days post injection (21-42 days, p<0.01). See FIG. 19.

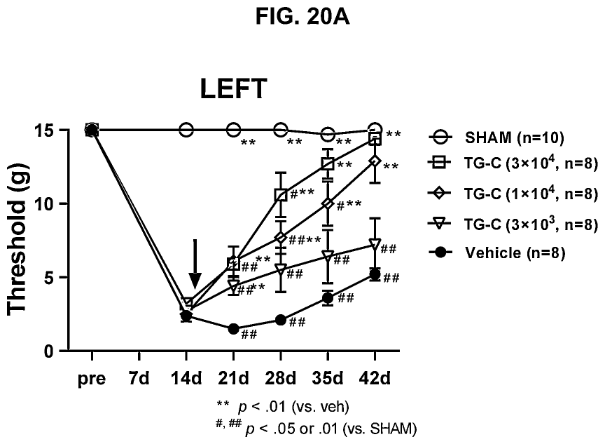

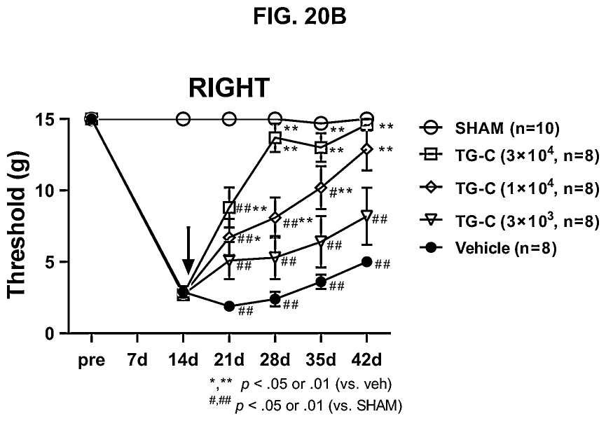

The results of the withdrawal test shown in FIGS. 20A-20B. As can be seen in these figures, the intradiscal injection of high and intermediate dose of mixed cells significantly increased the withdrawal threshold in hindlimbs of the MIA-injected animals from 21 to 42 days post treatment injection compared to the vehicle-treated, whereas the application of low doses of mixed cells significantly recovered the withdrawal threshold in left hindlimb of the MIA-injected animals 21 days post injection (all days, p<0.01; right hindlimb at intermediate dose of mixed cells at 21 days, p<0.05).

### Example X—Dose-Dependent Efficacy of Mixed-Cell Treatment for Alleviating Discogenic Pain in Monosodium Iodoacetate-Induced Discogenic Low Back Pain Model

All procedures were carried out as described in Example IX, except that the following doses of mixed cells were used: 1.0×10⁴ total cells, 3.0×10⁴ total cells, and 5.0×10⁴ total cells comprising a mixture of hChonJ and hChonJ #7 at the ratio of 3 to 1 (all groups n=8).

Treatment materials are shown in Table IV.

TABLE IV
Treatment Materials

| | |
|---|---|
| Name | hChonJ |
| Ingredient | Human allogeneic chondrocytes |
| Dose | 1.5 × 10⁷ cells/mL in 1.65 mL |
| Storage | LN2 Vapor Phase |
| Name | hChonJ#7 |
| Ingredient | Irradiated GP2-293 cells expressing TGF-β1 |
| Dose | 1.5 × 10⁷ cells/mL in 0.65 mL |
| Storage | LN2 Vapor Phase |

Excipient

| | |
|---|---|
| Name | CS10 (CRYOSTOR ® CS10) |
| Manufacturer | BioLifeSolutions |
| Size | 100 mL |
| Storage | 2-8° C. |

Experimental Design

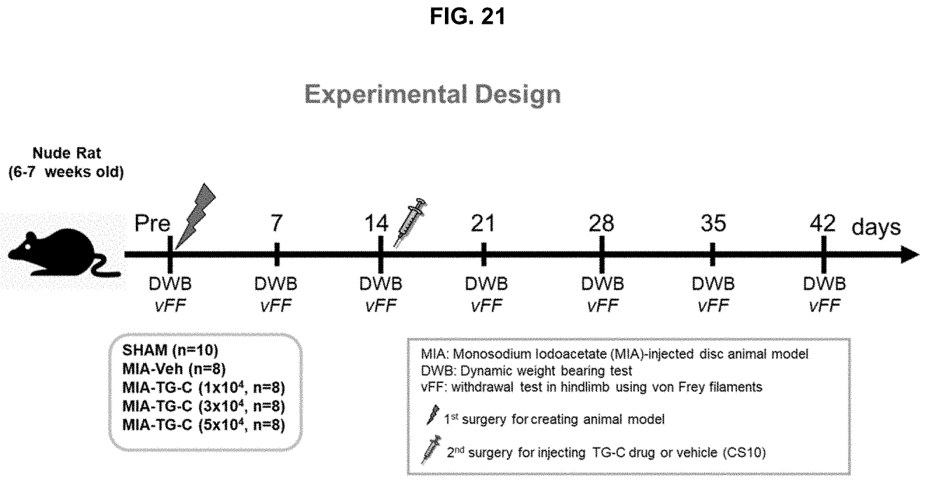

The experimental design is illustrated in FIG. 21.

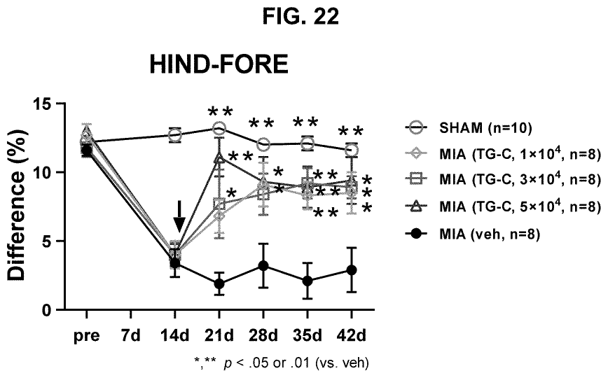

The results of the weigh bearing test are shown in FIG. 22. As can be seen in the figure, the LBP-associated behavior was strongly expressed 14 days after the intradiscal injection of MIA. The HIND-FORE weight bearing difference was from 11% to 4%; withdrawal threshold in hindlimb was from 15 g to 4 g. Intradiscal injections of mixed cells at all doses (1.0×10⁴ total cells, 3.0×10⁴ total cells, and 5.0×10⁴ total cells) significantly alleviated the LBP-associated behavior as compared to vehicle-treated animals.

Post hoc analysis indicates that MIA-injected animals treated with all doses of mixed cells (except for the dose of 1×10⁴ total cells at 21 days and 3×10⁴ total cells at 28 days) demonstrated significant recovery of dynamic weight bearing (increased HIND-FORE difference) compared to vehicle-treated animals 7 to 28 days post injection (p<0.01 or 0.01).

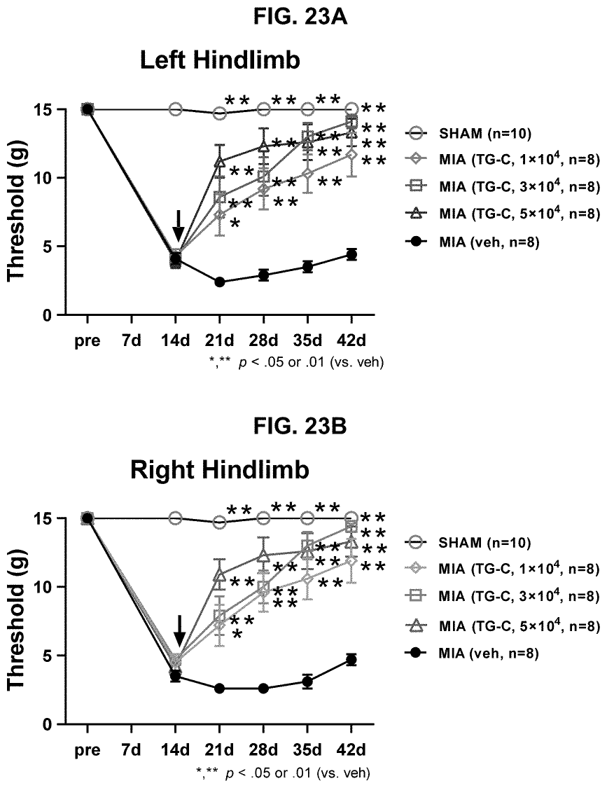

The results of the withdrawal test are shown in FIGS. 23A-23B. As can be seen in these figures, the intradiscal injection of all doses of mixed cells significantly increased the withdrawal threshold in left and right hindlimbs of MIA-injected animals from 7 to 28 days post injection compared to the vehicle-treated animals (all days, p<0.01; mixed cells at 1×10⁴ total cells on 21 days, p<0.05).

These experiments demonstrate that all doses of mixed cells have consistent suppressive effects on LBP including forelimbs-dependent weight load and lowered withdrawal threshold to hindlimb.

### Example XI—Calcium Imaging Test for Intervertebral Disc Degeneration in Mia-Induced Disc Degeneration Model

The study described in this Example evaluated whether mixed cell treatment is effective in alleviating sensitization of peripheral nerve system in MIA-induced LBP animal model.

Animals

Male Sprague-Dawley rats (270-290 g, n=18) were used for generating the MIA-induced animal model of discogenic LBP. Food and water were available ad libitum.

MIA-Induced Animal Model of Discogenic Low Back Pain

MIA-induced Animal Model of Discogenic Low Back Pain was generated as described in Example VI.

Dil-Labeling Neurons from Lumbar Intervertebral Discs

Rats were anesthetized with a mixture of alfaxalone (5 mg/kg) and medetomidine (0.25 mg/kg), and a midline ventral longitudinal incision was made in the supine position. The L4/5 and L5/6 disc were exposed under a microscope, intradiscal administration of 1,1′-dioctadecyl-3,3,3′,3′-tetramethylindocarbocyanine perchlorate (Dil, 2 μl) was cautiously performed.

Treatment

Mixed cells (3.0×10⁴ cells, mixture of human allogeneic chondrocytes and irradiated GP2-293 cells expressing TGF-β1 at the ratio of 3 to 1) or the vehicle (CRYOSTOR® CS10, BioLifeSolution, WA, USA) were administered to intradiscal space of L4/5 and L5/6 of rats in 2 μL with a Hamilton syringe (31 G needle).

Treatment and excipient compositions are listed in Table V.

TABLE V
Treatment Materials

| | |
|---|---|
| Name | hChonJ |
| Ingredient | Human allogeneic chondrocytes |
| Dose | 1.5 × 10⁷ cells/mL in 1.65 mL |
| Storage | LN2 Vapor Phase |
| Name | hChonJ#7 |
| Ingredient | Irradiated GP2-293 cells expressing TGF-β1 |
| Dose | 1.5 × 10⁷ cells/mL in 0.65 mL |
| Storage | LN2 Vapor Phase |

Excipient

| | |
|---|---|
| Name | CS-10 (CRYOSTOR ® CS-10) |
| Manufacturer | BioLifeSolutions |
| Size | 100 mL |
| Storage | 2-8° C. |

Treatment Preparation and Administration

Cell thawing and counting and treatment administration was performed as described in Example VI.

Intracellular Calcium Imaging

Lumbar dorsal root ganglions (DRG) (L1-4) of rats were collected 28 days after the surgery when Dil injection was performed. Rats were anesthetized with urethane (250 mg/kg; Sigma-Aldrich, St Louis, Mo., USA) and left and right lumbar DRG (L1-4) were rapidly sampled within 5-8 min after cutting portal vein. DRG neurons were enzymatically dissociated in Earle's balanced salt solution (EBSS; WELGENE, Seoul, South Korea) containing collagenase (19 μl/ml; Sigma-Aldrich), papain (12 μl/ml; Sigma-Aldrich), and CaCL (10 mM; 12 μl/ml; Sigma-Aldrich) for 35 min at 37° C. DRG neurons were dissociated by glass pipette trituration with three steps of decreasing tip pore size, then washed with DMEM culture medium (WELGENE) containing 10% fetal bovine serum and penicillin-streptomycin; Sigma-Aldrich) by centrifugation (two times at 1200 g for 2 min), and suspended in 200 μl culture media. The dissociated neuron suspension was transferred onto 12 mm circular glass coverslips (Paul Marienfeld, Germany) coated with poly D-lysine (Sigma-Aldrich). Dil-labeling was identified under a fluorescent microscope (filter, 540/25 nm of excitation, 605/55 nm of emission). The coverslips were loaded with Fura-2 AM (1 μl/ml, Invitrogen, CA, USA) in the DMEM culture medium and incubated for 30 min at 37° C. The coverslips were placed in a custom-built chamber (300 μl of bath volume) that was superfused with Locke solution (mM: 136 NaCl, 5.6 KCl, 1.2 MgCl₂, 2.2 CaCl₂, 1.2 NaH₂PO₄, 14.3 NaHCO, and 10 dextrose; pH 7.3-7.4 after aeration with 5% CO₂) by gravity. Changes in intracellular free Ca²⁺ in Dil-labeled neurons were measured by a digital camera (Zyla 5.5 sCMOS camera, Andor Technology, Northern Ireland) on the fluorescent microscope (1X71, Olympus, Tokyo, Japan) using a commercial program (Metamorph and Metafluor, Molecular Devices, USA). The flow rate of the bath solution was set at 1-2 ml/min. A field of targeted neurons was monitored by sequential dual excitation (340 nm and 380 nm), and the ratio images were acquired every 5 sec. The neurons on a coverslip were exposed to 1 μM capsaicin (Sigma-Aldrich) or 100 μM AITC (Sigma-Aldrich) for 20 sec, and then exposed to 50 mM KCl for 30 sec. The neurons were considered healthy only if they responded to 50 mM KCl with a rapid rise in intracellular Ca²⁺. After the intracellular Ca²⁺ measurement by fluorescent ratio images, a bright field image was also captured, and only small-sized DRG neurons (diameter of less than 20 μm) were included for analysis.

Experimental Design

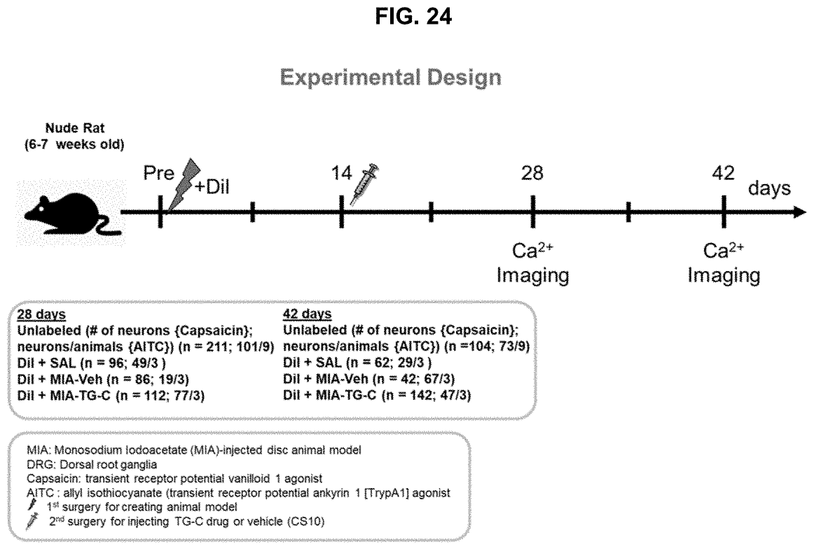

The experimental design of this study is illustrated in FIG. 24. The MIA-induced animal model of discogenic LBP was established as described above.

Calcium imaging test was performed 28 and 42 days after the first surgery (14 and 28 days after the second surgery for the injection of mixed cells or vehicle). The animals were divided into eight groups: unlabeled (number of neurons for capsaicin; neurons per animal for AITC: n=211; 101/9), Dil and saline (Dil+SAL) (n=96; 49/3), Dil, MIA and vehicle (Dil+MIA+VEH) (n=86; 19/3), and Dil, MIA and mixed cells (Dil+MIA+mixed cells) (n=112; 77/3) on day 14, and unlabeled (n=104; 73/9), Dil+SAL (n=62; 29/3), Dil+MIA+VEH (n=42; 67/3), and Dil+MIA+mixed cells (n=142; 47/3) on day 28.

Statistical Analysis

Data were expressed as means with standard error and evaluated using SPSS software version 28 (IBM corp., USA). Parametric or non-parametric statistics analysis of data was used depending on the pass of normal distribution testing. The peak normalized ratio (340/380) of calcium imaging was analyzed using a one-way ANOVA with a post hoc Tukey test. Differences between experimental conditions were considered statistically significant when p<0.05.

Results

The intradiscal application of mixed cells significantly decreases TrpV1 or TrpA1-dependent calcium influx in animals with MIA-induced LBP.

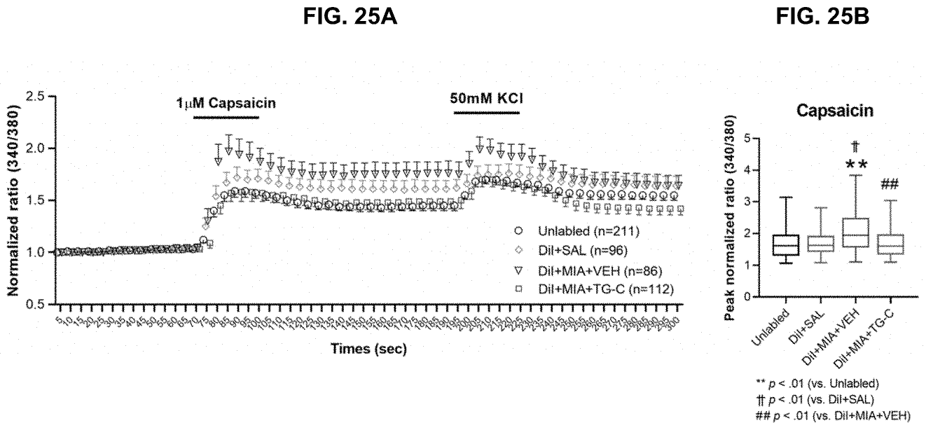

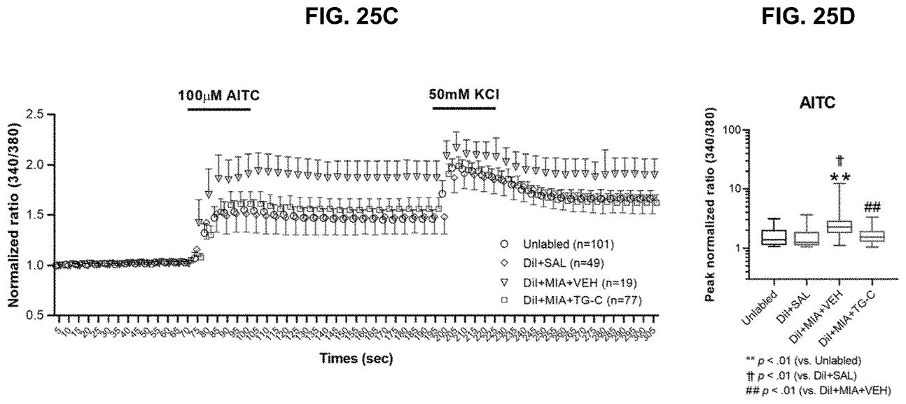

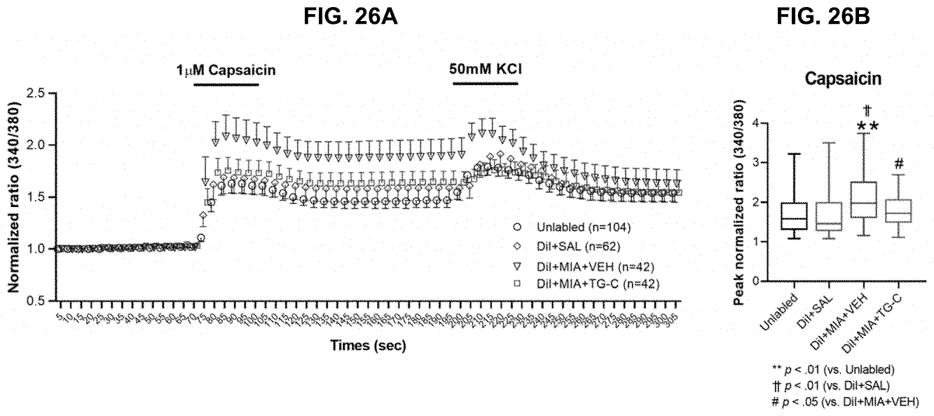

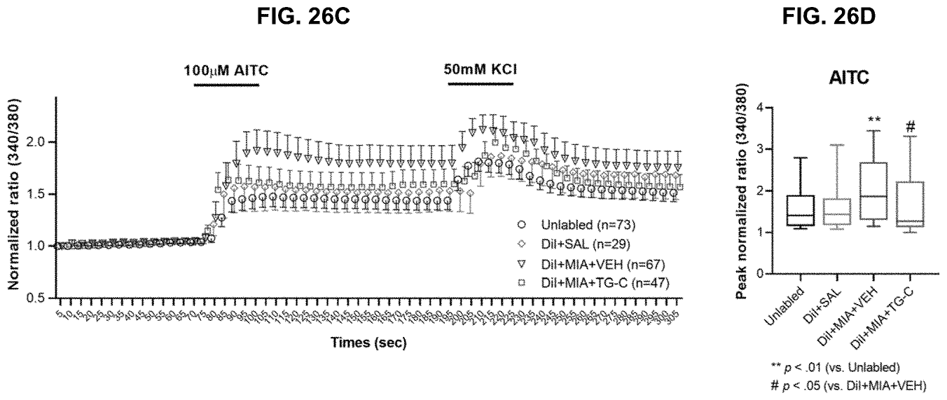

FIGS. 25A and 25C show normalized ratio (340/380; Fura-2AM) traces of calcium influx in primary cultured neurons of DRG 14 days post-Dil+MIA surgery. FIGS. 26A and 26C show normalized ratio (340/380; Fura-2AM) traces of calcium influx in primary cultured neurons of DRG 28 days post-Dil+MIA surgery. The peak normalized ratio of capsaicin (TrpV1 agonist) or AITC (TrpA1 agonist)-evoked calcium influx among unlabeled, Dil+SAL. Dil+MIA+VEH, and Dil+MIA+mixed cells groups were quantitatively examined.

Post hoc test indicates that Dil+MIA+VEH group showed significant increases in the peak normalized ratio of 1 μM capsaicin-evoked calcium influx compared to unlabeled and Dil+SAL group (all p<0.01 on 14 or 28 days) and of 100 μM AITC-evoked calcium influx compared to unlabeled and Dil+SAL group (vs. unlabeled and Dil+SAL, all p<0.01 on 14 days; vs. unlabeled, p<0.05 on 28 days). However, Dil+MIA+mixed cells group significantly recovered the peak normalized ratio of calcium influx in response to 1 μM capsaicin (p<0.01 at 14 days, p<0.05 at 28 days) or 100 pM AITC (p<0.01 at 14 days, p<0.05 at 28 days) compared to Dil+MIA+VEH group. See FIGS. 25B and 25D and FIGS. 26B and 26D, respectively.

The above calcium imaging studies showed that the intradiscal application of mixed cells created a strong analgesic effect in animals with MIA-induced LBP.

What is claimed is:

1. A method for preventing or treating chronic back pain in a subject in need thereof, comprising administering an effective amount of a composition comprising a mixed cell population to an intervertebral disc site of the subject, wherein the mixed cell population comprises a first mammalian cell comprising an exogenous nucleotide sequence encoding a protein having an intervertebral disc regenerating function and a second mammalian cell that does not comprise the exogenous nucleotide sequence and is a connective tissue cell.

2. The method according to claim 1, wherein the protein having an intervertebral disc regenerating function belongs to the TGF-β superfamily.

3. The method according to claim 2, wherein the protein of the TGF-β superfamily is a human or recombinant TGF-β1 protein.

4. The method according to claim 1, wherein the first mammalian cell is a human embryonic kidney cell or an epithelial cell, and the second mammalian cell is a chondrocyte.

5. The method according to claim 4, wherein the human embryonic kidney cell is modified to stably express TGF-β1 protein.

6. The method according to claim 5, wherein the human embryonic kidney cell or an epithelial cell is irradiated.

7. The method according to claim 4, wherein the chondrocyte is a non-disc chondrocyte or a juvenile chondrocyte.

8. The method according to claim 7, wherein the chondrocyte is a primed chondrocyte.

9. The method according to claim 8, wherein the chondrocyte is primed by incubation with a cytokine.

10. The method according to claim 9, wherein the cytokine is a member of the TGF-β superfamily.

11. The method according to claim 9, wherein the cytokine is TGF-β1 derived from the first mammalian cell expressing TGF-β1

12. The method according to claim 1, wherein the first and/or the second mammalian cell is allogeneic relative to the subject.

13. The method according to claim 1, wherein the subject has an intervertebral disc defect, the chronic back pain is a discogenic back pain, and the composition is administered by injection into the intervertebral disc defect site.

14. The method according to claim 13, wherein the mixed cell population contains a plurality of the first mammalian cells and a plurality of the second mammalian cells at a ratio of about 1 to 1-10.

15. The method according to claim 14, wherein the ratio is about 1 to 3.

16. The method according to claim 13, wherein the composition further comprises a cytokine.

17. The method according to claim 16, wherein the cytokine is a member of the TGF-β superfamily.

18. The method according to claim 13, wherein the composition further comprises a pharmaceutical carrier.

19. The method according to claim 18, wherein the pharmaceutical carrier comprises about 10 to 20% w/w dimethyl sulfoxide and about 1 to 5 w/w % saccharose.

20. The method according to claim 13, wherein the method reduces the discogenic back pain in the subject and/or reduces a back pain-associated behavior.

21. The method according to claim 20, wherein the method reduces TrpV1 or TrpA1-dependent calcium influx.

22. The method according to claim 13, wherein the method reduces sensitivity of the subject to the discogenic back pain.
